(19) United States
(12) Patent Application Publication
(10) Pub. No.: US 2024/0182864 A1
(43) Pub. Date: Jun. 6, 2024

(54) NOVEL CHEMICAL COMBINATIONS AND METHODS OF USE THEREOF, TOWARDS DIFFERENTIATION OF HUMAN PROGENITOR CELLS INTO FUNCTIONAL BETA CELLS

(71) Applicant: Salk Institute for Biological Studies, La Jolla, CA (US)

(72) Inventors: Haisong LIU, La Jolla, CA (US); Ronghui LI, La Jolla, CA (US); Hsin-Kai LIAO, La Jolla, CA (US); JUAN CARLOS IZPISUA BELMONTE, La Jolla, CA (US)

(21) Appl. No.: 18/551,986

(22) PCT Filed: Mar. 24, 2022
(86) PCT No.: PCT/US2022/021707

§ 371 Date: Sep. 22, 2023

Related U.S. Application Data
(60) Provisional application No. 63/165,441, filed on Mar. 24, 2021.

(51) Int. Cl. C12N 5/071

(52) U.S. Cl. CPC C12N 5/0676; C12N 2500/38; C12N 2501/01; C12N 2501/105; C12N 2501/11; C12N 2501/117; C12N 2501/12; C12N 2501/15; C12N 2501/155; C12N 2501/30; C12N 2501/41; C12N 2501/415; C12N 2501/72; C12N 2501/727; C12N 2506/02

(57) ABSTRACT

Compositions for generating a pancreatic beta-like cell population from a population of undifferentiated cells and methods of use thereof, are provided. The method is an 8 stage process interrupted by a priming step, and it includes; using a chemically defined protocol for the efficient generation of pancreatic progenitors (PPs); improved assembly PPs into 3D clusters; a priming step which uses a chemical/factor cocktail (PP-10C) to maintain 3D-PPs status and enhances their potential to differentiate into β cells ;and a 3-step differentiation protocol using select chemical cocktails that efficiently converts PP-10C-treated 3D-PPs into functional β cells.

The disclosed methods result in a population of functional β cells which expresses pancreatic cell markers selected from the group of c-peptide, the transcription factors NKX6.1, PDX1, PAX6, NEUROD1 and are INS+, and which do not express substantial levels Glucagon (GCG).

The functional β cells can be used to treat conditions such as diabetes.

### CROSS-REFERENCE TO RELATED APPLICATION

The present application claims priority to U.S. Application No. 63/165,441, filed Mar. 24, 2021, the disclosure of which are incorporated herein by reference in their entirety.

### FIELD OF THE INVENTION

This invention is generally directed to forward compositions and methods of producing mammalian cell populations that include a high proportion of pancreatic beta cells. Such cell populations are useful for treatment of diabetes. Materials and methods for the direct differentiation of stem cells, such as embryonic stem cells and induced pluripotent stem cells, into functional pancreatic beta cells are also provided.

### BACKGROUND OF THE INVENTION

Methodologies for guided differentiation of human pluripotent stem cells (hPSCs) into pancreatic βcells, with the goal of using these cells for cell replacement therapy to cure type I diabetes exist¹⁻¹¹. These current protocols usually use various growth factors or small molecules to simulate the development of pancreatic β cells, and gradually induce hPSC through various intermediate states (definitive endoderm, posterior foregut, pancreatic progenitor (PP), and endocrine progenitor (EP)) to generate insulin-producing cells¹⁻¹¹. However, the application potential of current methodologies remains limited because of several major issues.

First, the reported protocols for generating β cells (marked by NKX6.1+/INS+) from hPSCs are still not efficient, ranging from about 10% to about 40% efficiency in convesion^{2, 6-8, 11}. In order to reduce the cost of β-cell production, greater efficiency is necessary.

Second, the hPSC-derived β cell cultures are often heterogeneous and contain unwanted cell types that could possibly impede the maturation and function of βcells by secreting interfering factors. Although purification and re-aggregation of hPSC-derived βcells alleviates the negative influence and promotes beta cell maturation¹², such a method is not efficient and increases the overall cost associated with hPSC-derived β cell generation. In addition, the heterogeneous cell cultures pose a risk of teratoma formation after transplantations^{14, 15}. Dual-hormonal GCG+/INS+ cells, which are considered immature α cells, frequently emerge in β-cell differentiation cultures^{6, 9, 16, 17}. It still remains a challenge to reduce the presence of these GCG+/INS+ cells.

Third, current protocols have highly variable efficiencies depending on the origin of cell line manipulated. For example, Rezania, et al. generated ˜40% NKX6.1+/INS+ βcells from H1 human embryonic stem cells (hESCs), but only achieved ˜10% efficiency when applying the same protocol on a human induced pluripotent stem cell (hiPSC) line⁷.

There is thus a need for improved methods of generating functional β cells from progenitor cells.

It is therefore an object of the present invention to provide a method for functional β cells from progenitor cells.

It is also an object of the present invention to provide functional β cells from progenitor cells generated from progenitor cells.

It is still an object of the present invention to provide a method of treating diabetes in a subject need thereof.

### SUMMARY OF THE INVENTION

Compositions for generating a pancreatic beta-like cell population from a population of undifferentiated cells and methods of use thereof, are provided. The disclosed methods are used to produce cell populations that include a high proportion of pancreatic beta-like cells and/or mature pancreatic cells. The disclosed method includes the following stages of cell culture: 1) using a chemically defined protocol for the efficient generation of pancreatic progenitors (PPs): 2) an improved method for assembling PPs into 3D clusters, 3) a method that uses a 10-chemical/factor cocktail (PP-10C) to maintain 3D-PPs status and enhances their potential to differentiate into β cells (rather than unwanted by-products such as glucagon (GCG)+/insulin (INS)+ cells), and 4) a 3-step differentiation protocol (with combinations of signaling pathway regulators that have not been reported for each step) that efficiently converts PP-10C-treated 3D-PPs into functional βcells.

Thus, in one aspect, an improved method of generating PDX1⁺ NKX6.1⁺ progenitor cells is provided, which includes four stages of cell culture, wherein undifferentiated cells are cultured in cell culture medium supplemented with effective amounts of compounds as disclosed herein, resulting in improved efficiency of up to 80%, i.e., the 80% of cells in the cell resulting population are PDX1⁺ NKX6.1⁺.

Stage 1 (Definitive endoderm) includes culturing an undifferentiated cell population such as stem cells in cell culture medium supplemented with: (i) a wnt activator and (ii) a TGFβ family member such as Activin-A, for an effective amount of time to generate definitive endoderm (DE) cells. A preferred wnt activator is chir99021. The cells are maintained in this stage 1 for an effective amount of time, with the amount of the TGFβ family member in the cell culture medium used to culture the cells decreased over time.

At Stage 2 (Primitive gut tube), cells from stage 1 are cultured in cell culture medium supplemented with: (i) Keratinocyte growth factor (KGF), (ii) a TGFβ receptor (BMP) inhibitor and (iii) vitamin C. A preferred TGFβ receptor (BMP) inhibitor is dorsomorphine.

At Stage 3 (Posterior foregut), cells from stage 2 are cultured in cell culture medium supplemented with (i) KGF, (ii) a transforming growth factor (TGF)β receptor (BMP) inhibitor, (iii) vitamin C: (iv) a retinoic acid signaling activator, and (v) an inhibitor of sonic hedgehog signaling. A preferred TGFβ receptor (BMP) inhibitor is noggin.

At Stage 4 (Pancreatic Progenitor), cells from stage 3 are cultured in cell culture medium supplemented with (i) epidermal growth factor (EGF), (ii) a TGFβ receptor (BMP) inhibitor (iii) vitamin C and (iv) nicotinamide.

The cell culture from stage 1 to stage 4 is effective to induce conversion of undifferentiated cells into a population of PDX1⁺ NKX6.1⁺ pancreatic progenitor (PP) cells with improved efficiency, when compared to a cell culture scheme in which the cells at stage 1 are cultured at a constant concentration of the TGFβ family member in the cell culture medium.

Also provided herein is a method for improving the assembly of PPs into 3D clusters (PP-3D cluster stage). The method includes culturing PDX1⁺ NKX6.1⁺ PP cells in aggregation medium supplemented with aa RHO/ROCK (Rho-associated, coiled-coil containing protein kinase) inhibitor. The aggregation medium is cell culture medium supplemented with (i) EGF: (ii) a TGFβ receptor (BMP) inhibitor; (iii) vitamin C: (iv) a retinoic acid signaling activator: (v) an inhibitor of sonic hedgehog signaling: (vi) nicotinamide: (vii) thyroid hormone: (viii) γ-amino butyric acid (GABA and (ix) ZnSO4. The PDX1⁺ NKX6.1⁺ PP cells are cultured in the supplemented aggregation medium for an effective amount of time to form clusters.

Also provided herein is a method for priming PDX1⁺ NKX6.1⁺ PP cell clusters for improving differentiation into functional β cells. The method, referred to herein as Stage 5 (PP-10C-treated pancreatic progenitor), includes culturing PDX1⁺ NKX6.1⁺ PP cell clusters, in an aggregation medium supplemented with (i) a PKC activator and (ii) an ALK5 (TGFβ receptor 1) inhibitor. A preferred ALK5 inhibitor is RepSox and a preferred PKC activator is TPB ((2S,5S)-(E,E)-8-(5-(4-(trifluoromethyl)phenyl)-2,4-pentadienoylamino)benzolactam). PDX1+NKX6.1⁺ PP cell clusters cultured according to this aspect of the invention maintain expression of NKX6.1, at least after 4 days in culture, and are primed for improving differentiation into functional β cells.

Also provided is a method of differentiating PP-3D clusters into functional βcells. The method includes a three stage process, referred to herein as Stage 6, Stage 7, and Stage 8.

At stage 6 (Endocrine progenitor), primed PP-3D clusters are cultured in cell culture medium supplemented with (i) KGF: (ii) a TGFβ receptor (BMP) inhibitor: (iii) a cyclic AMP activator: (iv) a retinoic acid signaling activator: (v) an inhibitor of sonic hedgehog signaling: (vi) an ALK5 inhibitor: (vii) thyroid hormone and (viii) a PKC activator, for an effective amount of time to obtain NKX6.1⁺/NEUROD1⁺ cells.

At Stage 7 (Immature β cells), cells from stage 6 are cultured in a supplemented cell culture medium, for a period of time ranging from 6-14 days, preferably, for about 12 days. The cell culture medium is supplemented with factors selected from the group consisting of (i) Hepatocyte growth factor (HGF): (ii) a TGFβ receptor (BMP) inhibitor: (iii) an inhibitor of notch signaling: (iv) a retinoic acid signaling activator: (v) Insulin growth factor (IGF)1: (vi) an ALK5 inhibitor: (vii) thyroid hormone: (viii) a Fibroblast growth factor (FGF) inhibitor and (ix) ZnSO₄. Preferably cell culture medium used to culture the cluster is supplemented with factors (i), (iii), (iv), and (v) only during days 1-6 of cell culture and with factor (viii) is used to supplement cell culture medium only at days 3-6 of culture.

At stage 8, cells from stage 7 are cultured in supplemented cell culture medium for a period of time ranging from 8-14 days, preferably, for about 12 days. The cell culture medium is supplemented with factors selected from the group consisting of: (i) betacellulin: (ii) a NEUROD1 inducer: (iii) a G protein-coupled estrogen receptor 1 (GPER) agonist; (iv) a histone methyltransferase inhibitor: (v) an aurora kinase inhibitor; (vi) a ROCK inhibitor; and (vii) a pan-ErbB inhibitor. Preferably cell culture medium used to culture the cluster is supplemented with factor (vi) only during days 1-6 in culture and with factor (vii) only during days 7-12 in culture. Cell culture in combined stages 6-8 as disclosed is effective to convert PP-3D clusters into functional βcells, which express pancreatic cell markers selected from the group of c-peptide, the transcription factors NKX6.1, PDX1, transcription factor NEUROD1 and are insulin positive (INS+).

Also disclosed are functional βcells obtained according to the methods outlined in stages 1-4, PP-3D cluster and 4-8, as well as a population of cells including functional β-cells as described herein. In a preferred embodiment, the β-cells and population of cells including β-cells, do not express substantial levels of a hormone such as, Glucagon (GCG) or Somatostatin (SST).

The pancreatic beta-like cells and mature pancreatic cells so produced by the disclosed methods can be administered to a subject in need thereof, such as a subject diagnosed with diabetes.

### BRIEF DESCRIPTION OF THE DRAWINGS

FIGS. 1A-1L are graphs showing the efficient generation of pancreatic progenitor (PP) 3D-clusters. FIG. 1A is a scheme showing the generation of the NKX6.1-NLS-GFP reporter hPSC line, using a LoxP-flanked Puromycin (Puro) selection, a 2A-NLS-GFP was knocked-in and fused in frame to the end of the endogenous NKX6.1 coding sequence. The Puro cassette was later removed by CRE excision. The cells show nucleus-localized GFP expression when endogenous NKX6.1 gene is activated. FIG. 1B is a bright field image showing the morphology of hPSCs right before differentiation. FIG. 1C is a bright field image showing the morphology of hPSCs-derived definitive endoderm cells (at the end of stage 1). FIG. 1D includes graphs showing flow cytometry analysis of control fibroblast cells and stage-4 culture cells from three different batches: batch 1, batch 2, and batch 3. FIG. 1E is a schematic showing the new strategy for assembling PPs into 3D-clusters. The PPs were dissociated and re-aggregated by centrifuging in V-bottom 96-well plate to form cell pellets, and then form 3D-clusters after incubation. FIG. 1F is a graph of cell cytometry analysis, showing the high percentage of PPs (NKX6.1+/PDX1+) existing in H1 cell-derived PP 3D-clusters. FIG. 1G is a schematic illustrating Method-1: H1 cell-derived PP 3D-clusters were induced into β-cell stage, using the last three steps of R-protocol. Both H and I show the same. FIG. 1H is a graph of cell cytometry analysis showing that end-stage cell culture was comprised of high percentage of INS+NKX6.1− cells (mainly GCG+/INS+ cells) and a low number of βcells (NKX6.1+/INS+), according to Method-1: H1 cell-derived PP 3D-clusters were induced into β-cell stage, using the last three steps of R-protocol. FIG. 1I is a graph showing flow cytometry analysis of end-stage differentiation cells containing an unwanted GCG+/INS+ cell population generated by using Method 1. FIG. 1J is a graph showing cell cytometry for NKX6.1 and PDX1 in PP-10C-treated PP 3D-clusters, demonstrating that PP-10C can maintain PPs status with minor differentiation. FIG. 1K is a schematic illustrating Method-2: H1 cell-derived PP 3D-clusters were first treated by PP-10C and then induced into β-cell stage using the last three steps of the R-protocol. Both N and O show the same. FIG. 1L is a graph of cell cytometry analysis showing that the end-stage cluster were comprised of higher percentage of β cells (NKX6.1+/INS+) and reduced number of GCG+/INS+ cells, according to Method-2: H1 cell-derived PP 3D-clusters were first treated by PP-10C and then induced into β-cell stage using the last three steps of the R-protocol.

FIGS. 2A-2G are graphs showing the systematic screening for the differentiation of pancreatic progenitors (PPs) into β cells. FIG. 2A is a schematic showing the entire protocol established in this study and the developed strategy of inducing PP-10C-treated PPs into β cells. There are extra 5 differentiation stages from hPSCs to PP-10C-treated PPs, so these 3 late stages are named as stage 6, 7 and 8. Underlined chemicals/factors indicate chemicals/factors that have not been previously reported for inducing hPSCs into βcells. The combinations of chemicals/factors used in stage 6, 7 and 8 are termed as EP-8C, iβ-9C and Fβ-7C, respectively. During the process of our chemical screening, we also found other conditions in stage 6 that could induce high expression of NKX6.1 and NEUROD1, but these cells failed to efficiently become β cells following further differentiation. For instance, FIG. 2B shows flow cytometry analysis of PP-10C-treated PPs were incubated with Condition #1(Cocktail-A)), and Condition#2 (Cocktail-A+PP2 (pyrrolo-pyrimidine Src family kinase inhibitor, 5 μM)), for 4 days, followed by cell cytometry analysis for the expression of NKX6.1 and NEUROD1. Cocktail-A=ZnSO₄+Heparin+LDN(0.1 μM)+T3(1 μM)+RepSox(10 μM, high concentration)+GXISS (100 nM)+SANTI(0.25 μM)+RA(0.05 μM)+FSK(10 μM). FIG. 2C shows Cell cytometry analysis of H1 hES-derived stage-8 cells by using Method 3. FIG. 2D-2E show the results of flow cytometry analysis of PP-10C-treated PP 3D-clusters treated with different combinations of chemicals/factors listed in Table 2. To obtain improved condition for stage 6 (endocrine progenitor stage), PP-10C-treated PP 3D-clusters were treated with different combinations of chemicals/factors for 6 days, followed by a suboptimal later-stage condition treatment for 14 days. Cells were then analyzed for expression of NKX6.1 and INS using cell cytometry, showing that condition #6 (containing 8 chemicals/factors, termed as EP-8C) generated the most NKX6.1/INS+ cells. FIG. 2F shows cell cytometry analysis of stage-8 cells showing that Fβ-7C collectively induced (NKX6.1+/INS+) β cells from earlier stages, while subtracting some factors from Fβ-7C at stage 8 reduced the efficiency. FIG. 2G. shows cell cytometry analysis of stage-8 cells showed adding iβ-9C at stage 7 efficiently induced (NKX6.1+/INS+) β cells from earlier stages, while subtracting some factors from iβ-9C at stage 7 reduced the final efficiency

FIGS. 3A-3F are graphs showing that β cells were functional both in vitro and in vivo. FIG. 3A is a bar graph showing human C-peptide secretion from stage-7, stage-8 cells and primary human pancreatic islets in response to low (3.3 mM) and high (16.7 mM) glucose concentrations under static conditions. FIG. 3B is a bar graph showing results of human C-peptide secretion from stage-8 cells in response to low glucose (3.3 mM, n=5), Exendin-4 (with 3.3 mM glucose n=5), high glucose (16.7 mM, n=4), and 30 mM KCl (n=4). FIG. 3C is a bar graph showing the total insulin content of stage-8 cells and human islets. FIG. 3D is a graph showing that stage-8 cells reversed the hyperglycemia in STZ-induced diabetic NSG mice. STZ was administrated ˜5 weeks before cell transplantation. Tx, transplantation. N=4 for experimental group, and 3 for control groups. Transplanted fibroblasts were used as control. FIG. 3E is a bar graph showing human C-peptide levels measured after overnight fasting and 60 min following an i.p. glucose bolus at 5 weeks post-transplant of stage-8 end-point cells (n=3 mice for each differentiation-and-transplantation batch). Transplanted fibroblasts were used as control. N.D., not detectable FIG. 3F shows images showing the morphology of engraftments derived from different cells after transplantation in normal NSG mice, including stage-4 cells, stage-7 cells, stage-8 cells, and undifferentiated H1 hPSCs. The arrows indicate engraftments. K, kidney. Data are presented as mean+/−SEM for a, b, d and e, and mean+/−SD for c, respectively. P-values were determined by t-tests (two-sided) for a, b, d and e. N.D., not detectable. N.S., Not Significant. The estimated purity of primary human pancreatic islets was about 70%. FIG. 3G shows cell cytometry analysis of stage-8 cells from two human iPSC lines: human iPS line 1 (left panel) and line 2 (left panel). The NKX6.1+/INS+ cell population represents β cells.

FIGS. 4A-4D are graphs showing the efficiency of β cells formation using the strategy developed herein (FIG. 4A) in comparison with those reported in Peterson (FIG. 4B, reproduced from Peterson), Hogrebe (FIG. 4C, reproduced from Hogrebe), and Rezania (FIG. 4D, reproduced from Rezania).

FIGS. 5A-5D are graphs showing the static glucose-stimulated insulin secretion (GSIS) using the strategy developed herein (F FIG. 5A) in comparison with those reported in Peterson (FIG. 5B, reproduced from Peterson), Hogrebe (FIG. 5C, reproduced from Hogrebe), and Rezania (FIG. 5D, reproduced from Rezania).

FIGS. 6A-6D are graphs showing in vivo GSIS using the strategy developed herein (FIG. 6A) in comparison with those reported in Peterson (FIG. 6B, reproduced from Peterson), Hogrebe (FIG. 6C, reproduced from Hogrebe), and Rezania (FIG. 6D, reproduced from Rezania).

### DETAILED DESCRIPTION OF THE INVENTION

### I. Definitions

As used herein a “culture” means a population of cells grown in a medium and optionally passaged. A cell culture may be a primary culture (e.g., a culture that has not been passaged) or may be a secondary or subsequent culture (e.g., a population of cells that have been subcultured or passaged one or more times).

The term “defined” as used herein as applied to culture medium or storage medium or culture conditions indicates that the identity and the quantity of each component in the medium is known.

The term “pluripotent stem cell” (also referred to as “PSC”) as used herein refers to a cell having an ability to differentiate into any type of cell of an adult (pluripotency) and also having self-renewal capacity which is an ability to maintain the pluripotency during cell division. “PSCs” include embryonic stem cells (ESCs), which are derived from inner cell mass of blastocysts or morulae, including cells that have been serially passaged as cell lines. Embryonic stem cells, regardless of their source or the particular method used to produce them, can be identified based on their ability to differentiate into cells of all three germ layers. expression of at least Oct4 and alkaline phosphatase, and ability to produce teratomas when transplanted into immunodeficient animals. The term PSCs also includes induced PSCs (iPSCs), which are cells converted from somatic cells by a variety of methods, such as a transient overexpression of a set of transcription factors. A PSC may be a cell of any species with no limitation. and preferably a mammalian cell. It may be a rodent or primate cell. For example, it may be a monkey, mouse or a human pluripotent stem cell. The term “human pluripotent stem cells” or hPSCs includes human embryonic stem cells and human induced PSCs. Human embryonic stem cells may be obtained from established lines of human embryonic stem cells or human embryonic germ cells, such as, for example, the human embryonic stem cell lines H1, H7, and H9 (WiCell).

The term “isolated” or “purified” when referring to a population of β-cells means a β-cell population at least 50%, 55%, 60%, 65%, 70%, 75%, 80%, 85%, 90%, 95%, or 99% free of contaminating cells such as INS+/GCG+ bi-hormonal cells.

### II. Compositions

### A. Chemical Differentiation Agents

Compositions for use in an 8 stage process for efficient conversion of progenitor cells into a population of cells including βcells are disclosed. Each of the compounds described herein includes pharmaceutically acceptable salts thereof. Other compounds having these functions (i.e., functional analogs) are included within the scope of this disclosure. A “functional analog” as used herein means a compound that has a similar physical. chemical. biochemical, or pharmacological property as compared to another compound. Functional analogs may or may not have similar structures as compared to one another.

Compounds used in the disclosed methods include the following compounds/compounds with the following activities: a wnt activator, a TGFβ family member: growth factors such as KGF, EGF, HGF, IGF; a TGFβ receptor (BMP) inhibitor: vitamin C: an inhibitor of sonic hedgehog signaling: a retinoic acid signaling activator: nicotinamide; ZnSO4; thyroid hormone: GABA: a PKC activator; an ALK5 (TGFβ receptor 1) inhibitor; a gamma secretase inhibitor; a fibroblast growth factor (FGF) inhibitor: betacellulin; ii) a NEUROD1 inducer: a G protein-coupled estrogen receptor 1 (GPER) agonist; a histone methyltransferase inhibitor; an aurora kinase inhibitor: a ROCK inhibitor; and a pan-ErbB inhibitor.
(i) Wnt Activator

A wnt activator is preferably used in stage 1 of the disclosed methods. Suitable wnt activators that can be used in the disclosed methods include, but are not limited to chir99021 (6-[[2-[[4-(2.4-Dichlorophenyl)-5-(5-methyl-1/-imidazol-2-yl)-2-pyrimidinyl]amino]ethyl]amino]-3-pyridinecarbonitrile), SB216763, TWS119, CHIR98014, Tideglusib, SB415286, LY2090314, CHIR-98014, AZD1080, TDZD-8. BIO-acetoxime or wnt3a, with chir99021 being used in preferred embodiments.
(ii) TGFβ Family Member

A TGFβ family member is preferably used in stage 1 of the disclosed methods. Suitable TGFβ family members that can be used in the disclosed methods include, but are not limited to TGFβ, activin-A and Nodal, with Activin-A being used in preferred embodiments.
(iii) Growth Factors

A growth factor is preferably used in stages 2, 3, 4, PP-3D cluster, 6 and 7 of the disclosed methods. Suitable growth factors include, but are not limited to EGF, HGF, KGF and IGF.
iii) TGFα Receptor (BMP) Inhibitor

A TGFβ receptor (BMP) inhibitor is preferably used in stages 2-4, PP-3D cluster stage, and stages 6 and 7. Preferred TGFβ receptor (BMP) inhibitors are dorsomorphine, LDN193189 and noggin. Other TGFβ receptor (BMP) inhibitors are known in the art and are commercially available. Examples include, K 02288, ML347, DMH1, DMH2. LDN 214117 and LDN 212854.
(iv) A Retinoic Acid Signaling Activator

A retinoic acid signaling activator is preferably used in stage 3, PP-3D cluster stage, stage 6 and 7. Retinoic acid (RA) is used in preferred embodiments, although other retinoic acid signaling activators including, but not limited to TTNPB (4-[(E)-2-(5.6.7.8-Tetrahydro-5,5.8.8-tetramethyl-2-naphthalenyl)-1-propenyl]benzoic acid), EC23, AM580 (4-[(5.6,7.8-Tetrahydro-5.5,8,8-tetramethyl-2-naphthalenyl)carboxamido]benzoic acid). AM 80 and Ch55 (4-[(LE)-3-[3,5-bis(1, 1-Dimethylethyl)phenyl]-3-oxo-1-propenyl]benzoic acid), can also be used.
(v) Inhibitor of Sonic Hedgehog Signaling

An inhibitor of sonic hedgehog signaling is preferably used in stages 3, PP-3D cluster, and stage 6 of the disclosed methods, with SANTI ((4-Benzyl-piperazin-1-yl)-(3,5-dimethyl-1-phenyl-1H-pyrazol-4-ylmethylene)-amine) being used in preferred embodiments. SANT-I may be replaced with other inhibitors of sonic hedgehog signaling, including, but not limited to, KAAD-cyclopamine, cyclopamine, GANT61, Dynapyrazole A, Dynarrestin, Eggmanone, GANT58, Ciliobrevin A. PF 5274857 hydrochloride. SANT-2, and BMS-833923.
(vi) RHO ROCK (Rho-associated, coiled-coil Containing Protein Kinase) Inhibitor

A RHO/ROCK inhibitor kinase is preferably used in the PP-3D cluster stage of the disclosed methods as well we stage 8 of the disclosed methods. A preferred RHO/ROCK inhibitor is Y27632, however, other suitable RHO/ROCK inhibitors include, but are not limited to H1152, Fasudil GSK269962, Blebbistatin, HA1100 and RK11447 can also be used. A preferred a ROCK inhibitor used at stage 8 is H1152.
(vi) PKC Activator

A PKC activator is preferably used in stage 5 and 6 of the disclosed methods. A preferred PKC activator is TPB ((2S,5S)-(E,E)-8-(5-(4-(trifluoromethyl)phenyl)-2,4-pentadienoylamino)benzolactam). Other PKC activators which can be used in the disclosed methods, include, but are not limited to indolacltam V, SC9 (CAS 102649-78-5), SC10 (CAS 102649-79-6) , PMA (Phorbol-12-Myristate-13-Acetate), Prostratin; 8(S-hydroxy-(5Z, 9E, 11Z, 14Z)-eicosatetraenoic acid, and 1-Oleoyl-2-acetyl-sn-glycerol: 1-Oleoyl-2-acetylglycerol, (2/T, 4/T)-L-[(2L′,5U)-1,2,3,4,5,6-Hexahydro-5-(hydroxymethyl)-]-methyl-2-(1-methylethyl)-3-oxo-1,4-benzodiazocin-8-yl]-5-[4-trifluoromethyl)phenyl]-2,4-pentadienamide (TPPB), 5-Chloro-TV-(6-phenylhexyl)-1-naphthalenesulfonamide (SC-9), PDBu, Ingenol 3-angelate (PEP005), and Bryostatin 1.
(viii) ALK5 (TGFβ Receptor 1) Inhibitor

An ALK5 (TGFβ receptor 1) inhibitor is preferably used in stages 5, 6 and 7 of the disclosed methods. A preferred ALK5 inhibitor is RepSox. Other suitable ALK5 inhibitors include, but are not limited to GW788388, SB431542, and LY2109761.
(ix) Cyclic AMP Activator

A cyclic AMP activator is preferably used in stage 6 of the disclosed methods, with forskolin (FSK) or a forskolin analog being used in preferred embodiments, although other cAMP activator can be used, which include, but are not limited to dbcAMP, 8-Br-CAMP, prostaglandin E2 (PGE2), rolipram, and genistein.
(x) Notch Signaling Inhibitor

An inhibitor of notch signaling is preferably used in stage 7 of the disclosed methods. A preferred compound is γ-Secretase Inhibitor XX (GSIXX); although others such as DAPT, DBZ, BMS299897, Compound E, and L-685458 can replace GSIXX.
(xi) Fibroblast Growth Factor (FGF) Inhibitor

An FGF inhibitor is preferably used in stage 7 of the disclosed methods. A preferred FGF inhibitor is PD173074. Other suitable FGF inhibitors include, but are not limited to PD 161570, SU 5402, SU 5402, and SU 6668.
(xii) NEUROD1 Inducer

A NEUROD1 inducer is preferably used in stage 8 of the disclosed methods. A preferred neurod1 inducer is ISX-9 (N-Cyclopropyl-5-(2-thienyl)-3-isoxazolecarboxamide)
(xiii) G Protein-coupled Estrogen Receptor I (GPER) Agonist

A G protein-coupled estrogen receptor I (GPER) agonist is preferably used in stage 8 of the disclosed methods. A preferred GPER agonist is G-1

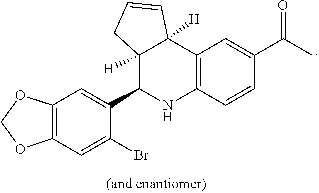 [SMILES: CC(=O)c1ccc2c(c1)[C@@H]1C=CC[C@@H]1[C@H](c1cc3c(cc1Br)OCO3)N2]

Other suitable PER agonists include, but are not limited to, ICI 182,780, and genistein.
(xiv) Aurora Kinase Inhibitor

An aurora kinase inhibitor is preferably used in stage 8 of the disclosed methods. A preferred aurora kinase inhibitor is ZM447439 (N-[4-[[6-Methoxy-7-[3-(4-morpholinyl)propoxy]-4-quinazolinyl]amino]phenyl]benzamide “ZM”). Other Aurora Kinase Inhibitors that can be used in the disclosed methods include, but are not limited to, VX 680, PF 03814735, and Hesperadin hydrochloride
(xix) Pan-ErbB Inhibitor

An Epidermal growth factor receptor (EGFR) family (pan-ErbB) inhibitor is preferably used in stage 8 of the disclosed methods. A preferred a pan-ErbB inhibitor is CI-1033 (Canertinib dihydrochloride). Other suitable pan-ErbB inhibitors that can be used in the disclosed methods include, but are not limited to, Dacomitinib, AZD8931, EKB-569, Lapatinib, PD 158780 and PKI-166.
(xx) Histone Methyltransferase Inhibitor

A histone methyltransferase inhibitor is preferably used in stage 8 of the disclosed methods. A preferred histone methyltransferase inhibitor, is Deazaneplanocin-A (Deza). Other suitable histone methyl transferase inhibitors include, but are not limited to Procainamide HCl, GSK503, SGC 0946 and SGI-1027.

Additional compounds that can be used in the disclosed methods include Vc (vitamin C), preferably used at stages 2, 3, and PP-3D cluster; ZnSO+, preferably used at the PP-3D cluster stage and stage 7: nicotinamide, preferably used at stage 4 and PP-3D cluster stage, thyroid hormone, preferably used at PP-3D cluster stage, stage 6 and 7: GABA, preferably used at the PP-3D cluster stage, and betacellullin (BTC), preferably used at stage 8.

A preferred combination of compounds for stage 6, termed EP-8C, include forskolin, RepSox, LDN, TPB, KGF, SANTI, RA, and T3.

A preferred combination of compounds for stage 7, termed iβ-9C, includes LDN, T3, RepSox, ZnS04, GSIXX, RA, HGF, IGF1, and PD173074.

A preferred combination of compounds for stage 8, termed Fβ-7C), includes BTC, ISX-9, G-1, Deza, ZM, H1152 and CI-1033.

The compound disclosed herein may be used at effective concentrations disclosed further herein. One of ordinary skill in the art can readily determine effective amounts of compounds disclosed herein. in view of the concentrations used and exemplified in the examples (concentrations incorporated herein by reference as particularly preferred embodiments).

The disclosed compounds and their equivalents are commercially available from companies such as Tocris Bioscience.

B. Chemically Induced Mature β-cells

Chemically induced functional β-cells produced according to the methods disclosed herein. In some embodiments the functional pancreatic beta cell is a human cell. Preferably, the cell expresses pancreatic cell markers selected from the group of c-peptide, the transcription factors NKX6.1, PDX1, NEUROD1⁺ and are INS⁺. Also disclosed is a population of cells including functional β-cells as described herein. In a preferred embodiment, the β-cells and population of cells including β-cells, do not express substantial levels of a hormone such as, Glucagon (GCG) or Somatostatin (SST), that serve as markers for cells other than pancreatic beta cells. A population of functional β-cells produced according to the methods disclosed herein, in some preferred embodiments has total insulin content of at least about 50, 55, or 60 ng per 10,000 cells, for example, at least about 61, 62, 63, 64, or 65, ng per 10,000 cells, measured for example, following acid-ethanol extraction and human ultrasensitive insulin ELISA kits.

Additionally, in an in vitro glucose stimulated C-peptide secretion assay shows that the stage 8 cells respond (measured by C-peptide secretion) to high-concentration (16.7 mM) glucose but not low-concentration (3.3 mM) glucose, and for human cells, under static conditions.

A population of cells obtained according to the disclosed methods may include INS+/GCG+ bi-hormonal cells, however, the population of cells is preferably at least about 50% NKX6.1+/INS+ cells, at least 60%, 65%, 70%, 75%, 80%, 85%, 90%, 95%, or 99% NKX6.1+/INS+ cells. For example, the population of cells can include up to about 80, 81, 82, 83, 84, 85% NKX6.1+/INS+ cells as measured for example, by Fluorescence-Activating cell Sorting (FACS), preferably obtained using the disclosed methods, without any subsequent cell enrichment step, after stage 8. Cell enrichment as used herein refers to the process of purifying cells for downstream application. This can be accomplished using antibody cocktails or by physically sorting out the cell populations of interest using a flow cytometer with sorting capability for example, fluorescence-activated cell sorting (FACS) or magnetic-activated cell sorting (reviewed in Suterman, et al. Scientific reports, Sei Rep 9, 227 (2019). https://doi.org/10.1038/s41598-018-36698-1.

### III. Method of Making

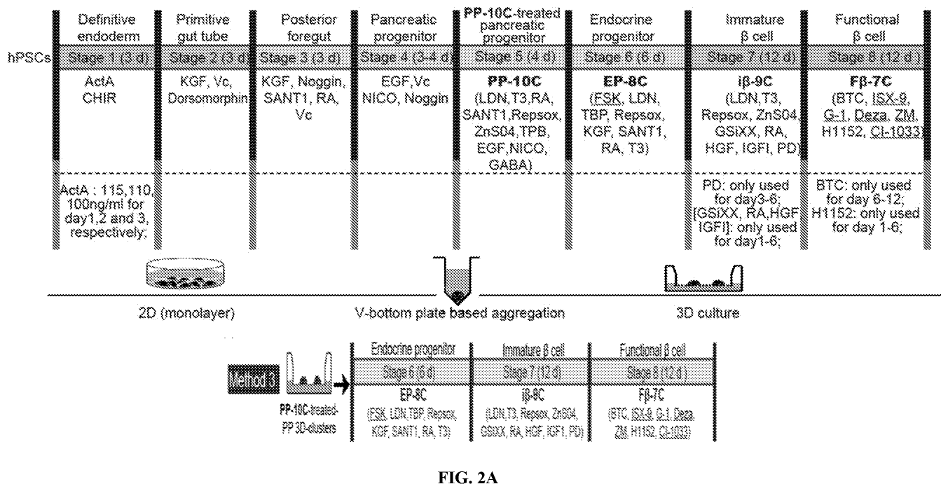

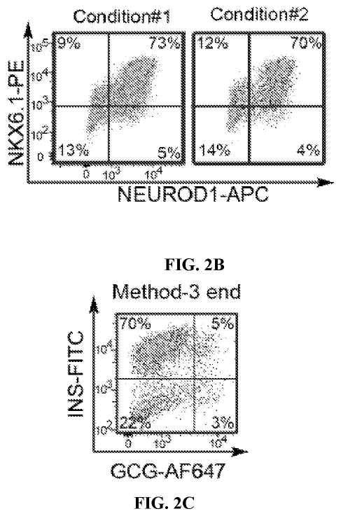

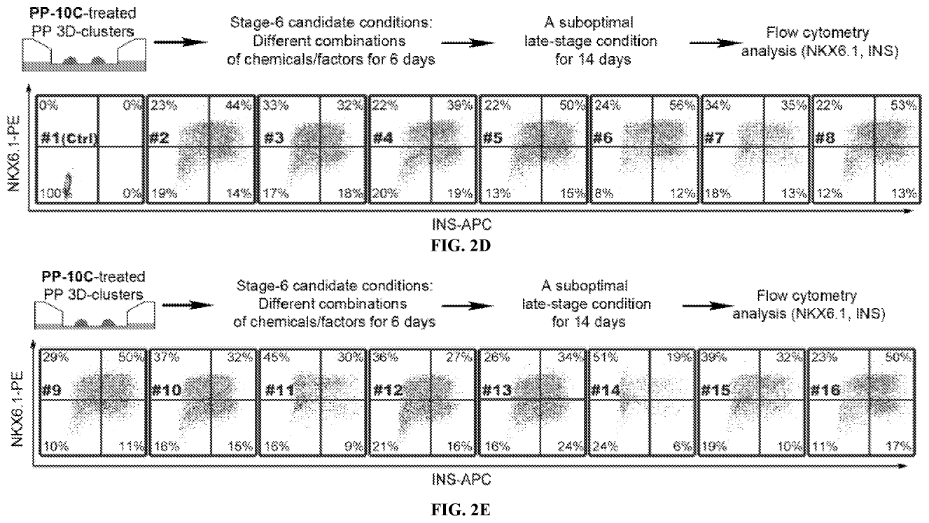

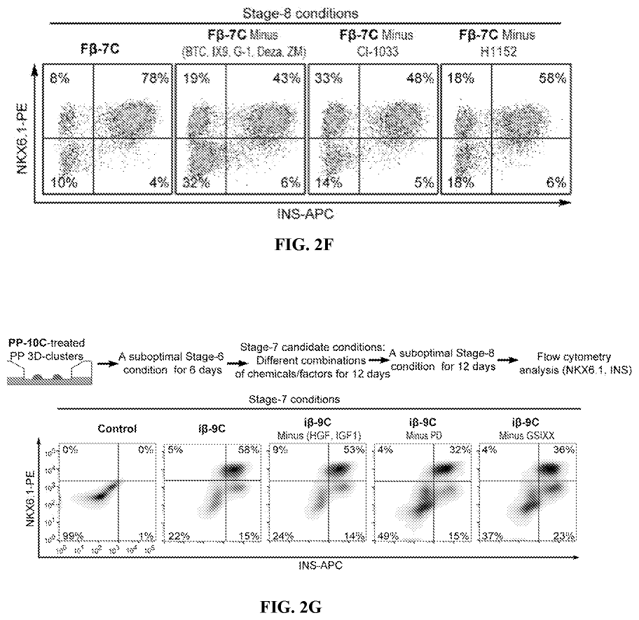

The disclosed method for converting undifferentiated cells into functional βcells is an 8 stage process (Stages 1 to 8; with stage 5 being preceded by an aggregation stage after Stage 4), a particularly preferred embodiment of which is shown in FIG. 2A (top panel). The disclosed method differ from prior disclosed methods for generating functional β cells from non-functional βcells, in that: (i) an improved final beta cell production efficiency is highest compared to any published protocols: (ii) the methods include a novel method for producing 3D-pancreatic progenitors clusters: (iii) uses novel combinations of chemicals in stage 5,6,7, and 8 are unique, including biological activities provided by compounds such as FSK at the Stage 6; ISX-9, G-1, Deza, ZM and CI-1033 at the Stage 8; (iv) the protocol is applicable to multiple cell lines (i.e., the protocol is cell line independent); and (v) the final beta cell population is of superior purity (measured by reduced proportion of unwanted GCG+/INS+ byproduct cells), compared to prior protocols: current other methods generate βcells with sub-optimal efficiency, and the effectiveness of these protocols varies when applying to different cell lines.

Undifferentiated cells that can be converted into functional βcells using the disclosed methods include but are not limited to stem cells such as human embryonic stem cells and induced pluripotent stem cells. Human embryonic stem cells may be obtained from established lines of human embryonic stem cells or human embryonic germ cells, such as, for example the human embryonic stem cell lines H1 and H9 (WiCell), H1-NKX6.1-GFP, or human induced pluripotent stem cells (hiPSC) cells such as integration-free hiPSC Induced pluripotent stem (IPS) cells are a type of pluripotent stem cell artificially derived from a non-pluripotent cell, typically an adult somatic cell. In an embodiment, in the practice of the present methods, the iPSCs can be derived from the patient who is the intended recipient of the generated cells, tissue or organ (i.e., may be autologous), or can be derived from an individual that is matched with the patient with respect to histocompatibility considerations. The iPSCs can be generated from any adult cells. For example, suitable cells include, but are not limited to, keratinocytes. dermal fibroblasts, leukocytes derived from peripheral blood, and cells obtained in urine. The iPSCs are generated by methods known in the art.
Cell Culture

For culturing of cells. any medium that is routinely used for culturing animal cells can be used, except that no growth factors or serum should be present or are added in the media. Examples of suitable culture media include mTeSRI, Essential 8 medium. BME, F-12, BGJb, MCDB131, CMRL 1066, Glasgow MEM, Improved MEM Zinc Option, IMDM, Medium 199, Eagle MEM, DMEM, Ham, RPMI 1640, and Fischer's media, but other similar media can also be used. Non-growth factor additives. such as antibiotics, B-27 supplement (with or without insulin). amino acids, salts, ascorbic acid and thioglycerol can be added to the media.

Incubation conditions for cell cultures are known in the art. For example, the conditions typically include culturing at a temperature of between about 32-40 ° C., for example, at least or about 32, 33, 34, 35, 36, 37, 38, 39 or 40 ° C. The CO2 concentration is generally about 1 to 10%, for example, about 2 to 7%, or about 5% or any range or value between 1 and 10%. The oxygen tension is adjusted to generally to provide normoxic conditions and is preferably about 20%.

The cells, starting with the pluripotent stem cells may be cultured on suitable substrates. For example suitable substrates include Matrigel, collagen IV, fibronectin, laminin, collagen, vitronectin, polylysine, iMatrix-511, iMatrix-521 and the like. These materials are commercially available and routinely used for cell culture.

Cells are cultured in cell culture medium supplemented with factors to provide a combination of specific biological activities as described further, below. The specific factors and concentrations disclosed below are preferred embodiments. However, for each disclosed factor. that factor can be replaced with a factor with the same biological activity, at an equivalent concentration as disclosed for the preferred compounds. Compounds that can be used to replace the preferred compounds below based on their disclosed biological activities are known in the art. and examples are provided in section II above.
Functional βCell Differentiation
Definitive Endoderm

In the first stage, undifferentiated cells, for example, human embryonic stem cells, are exposed to and cultured a differentiation medium (M12 medium) which is supplemented with a wnt activator and a TGFβ family member such as Activin-A for an effective amount of time to form DE cells, preferably, 3-5 days with the concentration of the TGFβ family in the cells culture mediums used to culture the cells reduced each day, relative to the previous day. A preferred cell culture protocol and cell culture supplementation is: MS12 medium supplemented with about 0.6 to 15 μM , preferably, 1-10 μM , more preferably, 1-6 μM, for example, 2, 3, 4 or 5 μM chir99021 and 23-600 ng/ml, preferably, 65-300, more preferably, 100-150 ng/ml, for example, 110, 115, 120, 2125 ng/ml Activin-A: day 2: same concentration of chir9902, with the concentration of Activin-A reduced by about 4-5%; thus, if the starting concentration of Activin-A is 115 ng/ml, it is reduced to 110 ng/ml; day 3: no supplementation with a wnt inhibitor, with the concentration of Activin-A further reduced by about by about 4-5%; thus if the concentration of Activin-A is 110 ng/ml on day 2, it is reduced to about 100 ng/ml.

A particular preferred MS12 medium is preferably prepared such that each 800 ml MS12 medium is prepared with MCDB131 medium (for example 744 m), glucose (3.2 ml ,45%, for example), fat-acid free BSA (20 ml 20%, for example), and sodium bicarbonate (16 ml ,7.5%, for example). One of ordinary skill in the art can substitute MCDB131 medium for other suitable cell culture media known in the art. The populations of cells provided herein can be at least 60% pure in the case of DE cells, for example, at least 60%. 70%, 80% or 90% DE cells.
Posterior Foregut

At stage 2, cells cultured as disclosed in stage one are cultured in MS12 medium supplemented with KGF [10-100 ng/ml, preferably, 25-75, more preferably 40-60 ng/ml, for example, 40, 45, 50, 55, to 60 ng/ml], vitamin C [0.05-2 mM, preferably, 0.1 to 1, and more preferably, 0.2-0.75 mM for example, 0.2, 0.25, 0.3, 0.35 mM] and a TGFβ receptor (BMP) inhibitor, preferably dorsomorphine [0.15-3.75, preferably, 0.3-1.875, more preferably, 0.45-1.5, for example, 0.55, 0.65, 0.75, 0.85, 0.95 μM], for 2-6 days, preferably 3-4 days.
Pancreatic Endoderm

At stage 3, cells cultured as disclosed in stage 2 are cultured for a period of time such as 2-6 days, preferably, 3-4 days, in cell culture medium such as DMEM, supplemented with retinoic acid signaling activator such as retinoic acid [0.4-10 μM, preferably, 0.8-5, more preferably, 1.2-3.5 μM, for example, 2 μM, 3 μM], a TGFβ receptor (BMP) inhibitor such as noggin [20-500 ng/ml, preferably, 50-250 ng /ml, more preferably, 100-150 ng/ml], an inhibitor of sonic hedgehog signaling such as SANTI [0.05-2 μM, preferably, 0.1 to 1, and more preferably, 0.2-0.75 μm for example, 0.2, 0.25, 0.3, 0.35 μM], Vitamin C [0.05-2 mM, preferably, 0.1 to 1, and more preferably, 0.2-0.75 mM for example, 0.2, 0.25, 0.3, 0.35 mM]. The cell culture medium preferably includes Pen/Strep (100×) and Glutamax (100×). Cells were fed with fresh medium every other day.
Pancreatic Progenitor (PP)

At stage 4, cells cultured as disclosed for stage 3 are cultured for a period of time such as 3-6 days, preferably, 3-4 days, in cell culture medium such as DMEM, supplemented with EGF [20-500 nM, preferably, 50-250, more preferably, 80-150, for example 80, 85, 90, 95, 100, 105, 110 ng/ml], Nicotinamide (NICO, 2-50 mM, preferably, 5-25 mM more preferably, 8-15 mM, for example 8, 8, 10, 11, 12, 13, 14 or 15 mM] a TGFβ receptor (BMP) inhibitor such as noggin [20-500 ng/ml, preferably, 50-250, more preferably, 80-150, for example 80, 85, 90, 95, 100, 105, 110 ng/ml] and Vitamin C [0.05-2 mM, preferably, 0.1 to 1, and more preferably, 0.2-0.75 mM for example, 0.2, 0.25, 0.3, 0.35 mM]. Cells were fed with fresh medium every other day.
PP Aggregation

At the aggregation stage, cells cultured as disclosed in stage 4 are preferably incubated in with Accutase for 10-15 min at 37° C. Dissociated single cells are re-suspended in aggregation medium supplemented with a RHO/ROCK (Rho-associated, coiled-coil containing protein kinase) inhibitor, with Y27632 [2-50 μM, preferably, 5-25 μM more preferably, 8-15 μM, for example 8, 8, 10, 11, 12, 13, 14 or 15 μM] being used in preferred embodiments. Aggregation medium is made of V4b-Medium+heparin [10 mg/ml]+ZnSO₄ [2-50 mM, preferably, 5-25 mM more preferably, 8-15 mM, for example 8, 8, 10, 11, 12, 13, 14 or 15 mM], a TGFβ receptor (BMP) inhibitor, with LDN193189 [20-500 nM, preferably, 50-250, more preferably, 80-150, for example 80, 85, 90, 95, 100, 105, 110 nM)] being used in preferred embodiments: T3 [0.2-5 μM, preferably, 0.5-2.5 μM, more preferably, 1-2 μM]: RA [0.01-0.25, preferably, 0.03-1, more preferably, 0.05-0.08, for example, 0.05, 0.06, 0.07 0.08 μm], an inhibitor of sonic hedgehog signaling, with SANTI [0.1-2 mM, preferably, 0.1 to 1, and more preferably, 0.2-0.75 mM for example, 0.2, 0.25, 0.3, 0.35 μm] being used in preferred embodiments: GABA [0.1-5 mM, preferably, 0.5-25.5 mM, more preferably, 1-1.5 mM]+EGF [20-500 ng/ml, preferably, 50-250 ng /ml, more preferably, 100-150 ng/ml]+NICO [2-50 mM, preferably, 5-25 mM more preferably, 8-15 mM, for example 8, 9, 10, 11, 12, 13, 14 or 15 mM]+Vitamin C [0.1-2 mM, preferably, 0.1 to 1 mM, and more preferably, 0.2-0.75 mM for example, 0.2, 0.25, 0.3, 0.35 mM]. V4b-Medium is made as disclosed in the examples. The cells are cultured in V-bottom 96-well plates as disclosed in the examples, until formation of clusters.
Primed PP Cell Priming

At Stage 5, PP-clusters for the aggregation stage are loaded on 6-well air-liquid interface trans-well plates and cultured for about 2-8 days, preferably, 3-6 days, for example, 3, 4, 5 or 6 days in stage-5 medium which is aggregation medium supplemented with PKC activator, with TPB [20-500 nM, preferably, 50 to 150 nM, more preferably, 80-150 nM, for example, 80, 85, 90, 95,100, 105, 110 nM] being used in preferred embodiments; and an ALK5 inhibitor, with RepSox [2-50 μM, preferably, 5-25 μM more preferably, 10-20 μM] being used in preferred embodiments. Cells were fed with fresh medium every other day. Cells treated as disclosed for this stage are referred to herein as PP-10C-treated pancreatic progenitor cells, and the treatment results in the formation of clusters.
Endocrine Progenitor

At Stage 6, clusters from stage 5 are cultured in Stage-6 medium was added for each well. Cells were fed with fresh medium every other day. Stage-6 medium which is B26-medium, supplemented with a cyclic AMP activator, Forskolin [2-50 μM, preferably, 5-25 μM more preferably, 10-20 μM) being used in preferred embodiments: T3 [0.2-5 μM, preferably, 0.5-2.5 μM, more preferably, 1-2 μM]. B26-medium is cell culture medium such as DMEM (preferably including B27(100×) and PS(100×)), supplemented with a TGFβ receptor (BMP) inhibitor, with LDN [100-2500 nM, preferably, 250-1500 nM, more preferably 500-1000 nM] being used in preferred embodiments: a PKC activator, with TPB [6-150 nM, preferably, 15-100 nM, more preferably, 20-40 nm, for example, 20, 25, 30, 35, 40, 45 nM] being used in preferred embodiments: an ALK5 inhibitor, with RepSox [0.2-5 μM, preferably, 0.5-2.5 μM, more preferably, 1-2 μM] being used in preferred embodiments: KGF [25 ng/ml)]: an inhibitor of sonic hedgehog signaling, with SANTI [0.1-2 mM, preferably, 0.1 to 1 mM, and more preferably, 0.2-0.75 mM for example, 0.2, 0.25, 0.3, 0.35 μM] being used in preferred embodiments, and a retinoic acid signaling activator, with RA [0.01-0.25, preferably, 0.03-1, more preferably, 0.05-0.08, for example, 0.05, 0.06, 0.07 0.08 μm] used in preferred embodiments.
Immature β-cells

At Stage 7, clusters from stage 6 are cultured in Stage-7 medium for about 12 days. Cells are fed with fresh medium every other day. Stage-7 medium was made of 4#-medium, supplemented with an inhibitor of notch signaling such as the γ-Secretase Inhibitor XX (GSIXX)[20-500 nM, preferably, 50 to 150 nM, more preferably, 80-150 nM, for example, 80, 85, 90, 95, 100, 105, 110 nM) only used to supplement medium for days 1-6); a retinoic acid activator (used only for days 1-6), with RA [0.01-0.25, preferably, 0.03-1, more preferably, 0.05-0.08, for example, 0.05, 0.06, 0.07 0.08 μM] , used in preferred embodiments: HGF [10-100 ng/ml, preferably, 25-75, more preferably 40-60 ng/ml, for example, 40, 45, 50, 55, to 60 ng/ml], only used to supplement medium for days 1-6): IGF1 [10-100 ng/ml, preferably, 25-75, more preferably 40-60 ng/ml, for example, 40, 45, 50, 55, to 60 ng/ml], only used to supplement medium for days 1-6): an FGF inhibitor with PD173074 [0.02-0.5 μM, preferably, 0.05-0.25, more preferably, 0.08-0.15 μM, for example, 0.08, 0.09, 0.1, 0.15 μM] used in preferred embodiments, only for days 3-6). 4#-medium=V4b-medium, supplemented with ZnSO₄ [2-50 μM, preferably, 5-25 μM more preferably, 8-15 μM, for example 8, 9, 10, 11, 12, 13, 14 or 15 μM]: optionally, Heparin [2-50 μM, preferably, 5-25 μM more preferably, 8-15 μM, for example 8, 9, 10, 11, 12, 13, 14 or 15 mg/ml]: a TGFβ receptor (BMP) inhibitor, with LDN [20-500 nM, preferably, 50 to 150 nM, more preferably, 80-150 nM, for example, 80, 85, 90, 95,100, 105, 110 nM] being used in preferred embodiments: T3 [0.2-5 μM, preferably, 0.5-2.5 μM, more preferably, 1-2 μM)]: and an ALK5 inhibitor, with RepSox [2-50 μM, preferably, 5-25 μM more preferably, 8-15 μM, for example 8, 9, 10, 11, 12, 13, 14 or 15 μM], being used in preferred embodiments.
Functional β-cells

At stage 8 clusters from stage 8 are cultured in Stage 8 medium for about 12 days, resulting in functional βcells. Clusters were rinsed in DF12 and transferred to a new trans-well. Cells were fed with fresh medium every other day. Stage-8 medium is made from M18#-medium, supplemented with 7 chemicals/factors (termed as Fβ-7C, including betacellulin [10 ng/ml], a NEUROD1 inducer, with ISX-9 [2-50 μM, preferably, 5-25 μM more preferably, 8-15 μM, for example 8, 9, 10, 11, 12, 13, 14 or 15 μM] used in preferred embodiments: a GPER agonist, with G-1 [0.2-5 μM, preferably, 0.5-2.5 μM, more preferably, 1-2 μM], used in preferred embodiments: a histone methyltransferase inhibitor, with Deazaneplanocin-A [0.2-5 μM, preferably, 0.5-2.5 μM, more preferably, 1-2 μM] used in preferred embodiments: an aurora kinase Inhibitor, with ZM447439 [ 0.1-2 mM, preferably, 0.1 to 1 mM, and more preferably, 0.2-0.75 mM for example, 0.2, 0.25, 0.3, 0.35 μM], ] used in preferred embodiments; a ROCK inhibitor (only used to supplement medium used for days 1-6), with H1152 [2-50 μM, preferably, 5-25 μM more preferably, 8-15 μM, for example 8, 9, 10, 11, 12, 13, 14 or 15 μM], used in preferred embodiments: and a pan-ErbB inhibitor (only used to supplement medium used for days 7-12), with CI-1033 [0.2-5 μM, preferably, 0.5-2.5 μM, more preferably, 1-2 μM] used in preferred embodiments.

Functional β cells obtained after stage 8 can be further purified/isolated using known cell sorting methodologies such as fluorescence-activated cell sorting (FACS), magnetic-activated cell sorting (Suterman, et al. Scientific reports, Sci Rep 9, 227 (2019). https://doi.org/10.1038/s41598-018-36698-1.). Cells can be cryopreserved for storage according to known methods. Media for preservation of cells are known in the art, for example, CRYO-GOLD™ (cryopreservation medium and CrosStor® (cyropreservation freeze media) designed to mitigate temperature-induced molecular cell stress responses during freezing and thawing. All CryoStor products are pre-formulated with USP grade DMSO, a permeant solute cryoprotective agent which helps mitigate damage from the formation of intracellular ice. CryoStor is offered in several packages and pre-formulated with DMSO in final concentrations of 2%, 5%, and 10%. A preferred medium for cell preservation includes 5-10% DMSO, for example, CryoStorR CS10 (a uniquely formulated serum-free, animal component-free, and defined cryopreservation medium containing 10% dimethyl sulfoxide (DMSO)). Additionally, cryoprotctants/cyroprotectant additives which can be include in a cell composition (for cryopreservation) are known in the art and include, but are not limited to dimethylsulfoxide (DMSO), thylene glycol (EG), antioxidants such as taurine, Metformin, gamma amino butyric acid (GABA), etc (Kojayan, et al., Systematic review of islet cryopreservation, Islets, 10:1. 40-49. DOI: 10.1080/19382014 2017.1405202). For example, cells may be suspended in a “freeze medium” such as cell culture medium containing 15-20% fetal bovine serum (FBS) and 7-10% DMSO, with or without 5-10% glycerol, at a density, for example, of about 4-10×10⁶ cells/ml. The cells are dispensed into glass or plastic vials, which are then sealed and transferred to a freezing chamber of a programmable or passive freezer. The optimal rate of freezing may be determined empirically. For example, a freezing program that gives a change in temperature of −1 ° C./min through the heat of fusion may be used. Once vials containing the cells have reached −80° C., they are transferred to a liquid nitrogen storage area.

Particularly preferred embodiments of each of stages 1-8 and the aggregation stage are exemplified in the Examples.

### IV. Method of Using

The disclosed functional βcells obtained according to the disclosed methods can be used in cell therapy, for example to treat a patient suffering from, or at risk of developing, diabetes. The diabetes can, for example, be type 1, type 2, or type 1.5 diabetes. The patient is preferably a mammalian subject such as a human patient, a domestic animal (for example a dog), a zoo animal or a laboratory animal.

Cells may be implanted into an appropriate site in a recipient. The implantation sites include, for example, the liver, natural pancreas, renal subcapsular space, omentum, peritoneum, subserosal space, intestine, stomach. or a subcutaneous pocket. The amount of cells used in implantation depends on a number of various factors including the patient's condition and response to the therapy, and can be determined by one skilled in the art. The number of cells for transplantation may be used from 1 million to 20 million and all integer values there between. In embodiments, the number of cells used may be 1, 2, 5, 7.5, 10, 12.5, 15, 17.5 and 20 million cells. In an example, the differentiated beta-cell lineage cells may be implanted as dispersed cells or formed into clusters that may be infused into the hepatic portal vein. Alternatively, cells may be provided in biocompatible degradable polymeric supports, porous non-degradable devices or encapsulated to protect from host immune response. Support materials suitable for use for implantation of cells of the present disclosure include tissue templates, conduits, barriers, and reservoirs, such as those used for tissue repair. For example, synthetic and natural materials in the form of foams, sponges. gels, hydrogels, textiles, and nonwoven structures may be used.

The cells to be administered can be incorporated into a three-dimensional support. The cells can be maintained in vitro on this support prior to implantation into the subject. Alternatively, the support containing the cells can be directly implanted in the subject without additional in vitro culturing. The support can optionally be incorporated with at least one pharmaceutical agent that facilitates the survival and function of the transplanted cells. Support materials suitable for use include tissue templates, conduits, barriers, and reservoirs useful for tissue repair. In particular, synthetic and natural materials in the form of foams, sponges, gels, hydrogels, textiles, and nonwoven structures, which have been used in vitro and in vivo to reconstruct or regenerate biological tissue, as well as to deliver chemotactic agents for inducing tissue growth, are suitable for use in practicing the methods described herein. See, for example, the materials disclosed in U.S. Pat. Nos. 5,770,417, 6,022,743, 5,567,612, 5,759,830, 6,626,950, 6,534,084, 6,306,424, 6,365,149, 6,599,323, 6,656,488, U.S. Published Application 2004/0062753 A1, U.S. Pat. Nos. 4,557,264 and 6,333,029, each of which is specifically incorporated by reference herein in its entirety.

To enhance further differentiation, survival or activity of the implanted cells in a subject. additional factors, such as growth factors, antioxidants or anti-inflammatory agents, can be administered before. simultaneously with, or after the administration of the cells.

The disclosed cells can also be used as a research tool for diabetes drug screening and beta-cell research.

The disclosed compositions and methods can be further understood through the following enumerated paragraphs or embodiments.

1. A method of generating cells of pancreatic beta lineage from pluripotent stem cells for example, embryonic stem cells or induced pluripotent stem cells comprising:

(a) culturing the pluripotent stem cells in a cell culture medium comprising one or more compounds with biological activities selected from the group consisting of: (i) a Wnt signaling activator, and (ii) a BMP signaling inhibitor to obtain a population of definitive endoderm (DE) cells:

(b) culturing the DE cells to cell culture medium comprising one or more compounds selected from the group consisting of. i) a growth factor, (ii) a TGFβ receptor (BMP) inhibitor and (iii) vitamin C to obtain primitive gut tube (PGT) cells:

(c) culturing the PGT cells in cell culture medium comprising one or more compounds selected from the group consisting of: (i) a growth factor, (ii) a transforming growth factor (TGF)β receptor (BMP) inhibitor, (iii) vitamin C: (iv) a retinoic acid signaling activator. and (v) an inhibitor of sonic hedgehog signaling: to form posterior gut (PG) cells:

(d) culturing PG cells in cell culture medium comprising one or more compounds selected from the group consisting of: (i) a growth factor, (ii) a TGFβ receptor (BMP) inhibitor (iii) vitamin C and (iv) nicotinamide to obtain pancreatic progenitor (PP) cells:

(e) culturing the PP cells in aggregation medium to form PP-3D clusters,
wherein the aggregation medium is cell culture medium comprising one or more compounds selected from the group consisting of: i) a growth factor: (ii) a TGFβ receptor (BMP) inhibitor: (iii) vitamin C: (iv) a retinoic acid signaling activator: (v) an inhibitor of sonic hedgehog signaling: (vi) nicotinamide: (vii) thyroid hormone: (viii) γ-amino butyric acid (GABA and (ix) ZnSO4; and

wherein the aggregation medium comprises a RHO/ROCK (Rho-associated, coiled-coil containing protein kinase) inhibitor;

(f) culturing the PP-3D clusters in aggregation medium comprising one or more compounds selected from the group consisting of: (i) a PKC activator and (ii) an ALK5 (TGFβ receptor 1) inhibitor, to form primed PP cells:

(g) culturing the primed PP cells in cell culture medium comprising one or more compounds selected from the group consisting of: (i) a growth factor: (ii) a TGFβ receptor (BMP) inhibitor: (iii) a cyclic AMP activator: (iv) a retinoic acid signaling activator: (v) an inhibitor of sonic hedgehog signaling: (vi) an ALK5 inhibitor: (vii) thyroid hormone and (viii) a PKC activator. to form NKX6.1⁺/NEUROD1⁺ endocrine progenitor (EP) cells:

(h) culturing the EP cells in cell culture medium supplemented with one or more compounds selected from the group consisting of (i) a growth factor: (ii) a TGFβ receptor (BMP) inhibitor: (iii) an inhibitor of notch signaling: (iv) a retinoic acid signaling activator: (v) an ALK5 inhibitor: (vi) thyroid hormone: (vii) a Fibroblast growth factor (FGF) inhibitor and (ix) ZnSO₄, to form immature β cells (IBC); and

(i) culturing the IBC in cell culture medium comprising one or more compounds selected from the group consisting of: (i) betacellulin: (ii) a NEUROD1 inducer; (iii) a G protein-coupled estrogen receptor 1 (GPER) agonist: (iv) a histone methyltransferase inhibitor; (v) an aurora kinase inhibitor: (vi) a ROCK inhibitor; and (vii) a pan-ErbB inhibitor.

2. The method of paragraph 1, wherein the growth factor is selected from the group consisting of epidermal growth factor (EGF), hepatocyte growth factor (HGF), keratinocyte growth factor (KGF) and insulin-like growth factor (IGF)

3. The method of paragraph 1 or 2, wherein the Wnt signaling activator is selected from the group consisting of CHIR99021. SB216763, TWS119, CHIR98014, Tideglusib, SB415286, LY2090314, CHIR-98014, AZD1080, TDZD-8 and wnt3a.

4. The method of any one of paragraphs 1-3, wherein the TGFβ family member is Activin-A or nodal.

5. The method any one of paragraphs 1-4, wherein the TGFβ receptor (BMP) inhibitor signaling inhibitor is selected from the group consisting of dorsomorphin, noggin, LDN193189, K 02288, ML347, DMH1, DMH2, LDN 214117 and LDN 212854.

6. The method of any one of paragraphs 1-5, wherein the sonic hedgehog signaling inhibitor is selected from the group consisting of SANTI. KAAD-cyclopamine. cyclopamine. GANT61, and BMS-833923.

7. The method of any one of paragraphs 1-5, wherein the PKC activator is selected from the group consisting of TPB ((2S,5S)-(E,E)-8-(5-(4-(trifluoromethyl) phenyl)-2,4-pentadienoylamino)benzolactam); (2/T, 4/T)-L-[(2L′,SU)-1,2,3,4,5,6-Hexahydro-5-(hydroxymethyl)-l-methyl-2-(1-methylethyl) -3-oxo-1,4-benzodiazocin-8-v1]-5-[4-(trifluoromethyl)phenyl]-2.4-pentadienamide (TPPB): 5-Chloro-TV-(6-phenylhexyl)-1-naphthalenesulfonamide (SC-9): PDBu; Ingenol 3-angelate (PEP005), and Bryostatin.

8. The method of any one of paragraphs 1-7, wherein the retinoic acid activator is selected from the group consisting of retinoic acid. TTNPB (4-[(E) -2-(5,6.7,8-Tetrahydro-5.5,8,8-tetramethyl-2-naphthalenyl)-1-propenyl]benzoic acid), EC23, AM580 (4-[(5.6,7.8-Tetrahydro-5,5,8.8-tetramethyl-2-naphthalenyl)carboxamido]benzoic acid). AM 80 and Ch55 (4-[(1E)-3-[3.5-bis(1.1-Dimethylethyl)phenyl]-3-oxo-1-propenyl]benzoic acid).

9. The method any one of paragraphs 1-8, wherein the ALK5 inhibitor is selected from the group consisting of RepSox, GW788388, SB431542, and LY2109761.

10. The method any one of paragraphs 1-9, wherein the RHO/ROCK (Rho-associated, coiled-coil containing protein kinase) inhibitor is selected from the group consisting of Y27632, H1152, Fasudil GSK269962, Blebbistatin, HA1100 and RK11447.

11. The method any one of paragraphs 1-10, wherein the cyclic AMP activator is selected from the group consisting of forskolin, dbcAMP, 8-Br-CAMP, prostaglandin E2 (PGE2), rolipram, and genistein.

12. The method any one of paragraphs 1-11, wherein the FGF inhibitor is selected from the group consisting of PD173074, PD 161570, SU 5402, SU 5402, and SU 6668.

13. The method any one of paragraphs 1-12. wherein the aurora kinase inhibitor is selected from the group consisting of ZM447439, VX 680, PF 03814735, and Hesperadin hydrochloride

14. The method any one of paragraphs 1-12, wherein the NEUROD1 inducer inhibitor is ISX-9 (N-Cyclopropyl-5-(2-thienyl)˜3-isoxazolecarboxamide)

15. The method any one of paragraphs 1-13. wherein the GPER agonist is selected from the group consisting of G-1, ICI 182,780, and genistein

16. The method any one of paragraphs 1-14, wherein the histone methyltransferase inhibitor is selected from the group consisting of Deazaneplanocin-A, Procainamide HCl, GSK503, SGC 0946 and SGI-1027.

17. The method any one of paragraphs 1-15, wherein the pan-ErbB is selected from the group consisting of Canertinib dihydrochloride, Dacomitinib, AZD8931, EKB-569, Lapatinib, PD 158780 and PKI-166.

18. The method of any one of paragraphs 1-17, wherein the cell culture medium is further supplemented with one or more compounds selected from the group consisting of vitamin C, ZnSO₄, nicotinamide, thyroid hormone, GABA and betacellullin

19. The method of any one of paragraphs 1-18, wherein the cell culture medium comprises a compound selected from the group consisting of LDN, T3, RepSox, ZnS04, GSIXX, RA, HGF, IGF1, and PD17307.

20. The method of any one of paragraphs 1-19, wherein the cell culture medium comprises a compound selected from the group consisting of BTC, ISX-9, G-1, Deza, ZM, H1152 and CI-1033.

21. The method of any one of paragraphs 1-20, wherein the cell culture medium further comprises about, 1-10 μM, more preferably, 1-6 μM, for example, 2, 3, 4 or 5 μM chir99021 and/or 23-600 ng/ml, preferably, 65-300, more preferably, 100-150 ng/ml, for example, 110, 115, 120, 125 ng/ml Activin-A.

22. The method of any one of paragraphs 1-21, comprising a growth factor such as KGF, at concentration of about 10-125 ng/ml, preferably, 25-75, more preferably 40-60 ng/ml, for example, 30, 35, 40, 45, 50, 55, to 60 ng/ml, EGF , at a concentration of about 20-500 nM, preferably, 50-250, more preferably, 80-150, for example 80, 85, 90, 95, 100, 105, 110 ng/ml]; HGF [10-100 ng/ml, preferably, 25-75, more preferably 40-60 ng/ml, for example, 40, 45, 50, 55, to 60 ng/ml], only used to supplement medium for days 1-6: or IGF1 [10-100 ng/ml, preferably, 25-75, more preferably 40-60 ng/ml, for example, 40, 45, 50, 55, to 60 ng/ml], only used to supplement medium for days 1-6); and/or TGFβ receptor (BMP) inhibitor such as dorsomorphine at a concentration of about 0.15-3.75, preferably, 0.3-1.875, more preferably, 0.45-1.5, for example, 0.55, 0.65, 0.75, 0.85, 0.95 μm, or such as noggin at a concentration of about 20-500 ng/ml, preferably, 50-250 ng /ml, more preferably, 100-150 ng/ml; or LDN193189 at a concentration of about 20-2500 nM, for example, at a concentration between about 50-250, between about 80-150, for example 80, 85, 90, 95, 100, 105, 110 nM: between about 250-1500 nM, between about 500-1000 nM, etc.

23. The method of any one of paragraphs 1-22, wherein the cell culture medium further comprises one or more of vitamin C, at a concentration of about 0.05-2 mM, preferably, 0.1 to 1, and more preferably, 0.2-0.75 mM for example, 0.2, 0.25, 0.3, 0.35 mM: retinoic acid signaling activator such as retinoic acid [0.4-10 μM, preferably, 0.8-5, more preferably, 1.2-3.5 μM, for example, 2μM, 3 μM: Nicotinamide, at a concentration of about 2-50 mM, preferably, 5-25 mM more preferably, 8-15 mM, for example 8, 8, 10, 11, 12, 13, 14 or 15 mM; ZnSO₄, at a concentration of about 2-50 mM, preferably, 5-25 mM more preferably, 8-15 mM, for example 8, 8, 10, 11, 12, 13, 14 or 15 mM: T3 at a concentration of about 0.2-5 μM, preferably, 0.5-2.5 μM, more preferably, 1-2 μM μM]: RA [0.01-0.25, preferably, 0.03-1, more preferably, 0.05-0.08, for example, 0.05, 0.06, 0.07 0.08 μm: GABA , at a concentration of about 0.1-5 mM, preferably, 0.5-25.5 mM, more preferably, 1-1.5 mM: betacellulin, at a concentration of about 2-50 ng/ml, preferably, 5-25 ng/ml, more preferably about 8-15 ng/ml.

24. The method of any one of paragraphs 1-23, wherein the cell culture medium further comprises a RHO/ROCK (Rho-associated, coiled-coil containing protein kinase) inhibitor, such as Y27632 at a concentration of about 2-50 M, preferably, 5-25 μM more preferably, 8-15 μM, for example 8, 8, 10, 11, 12, 13, 14 or 15 μM.

25. The method of any one of paragraphs 1-24, wherein the cell culture medium further comprises, a PKC activator, such as TPB at a concentration of about 6-500 nM, for example, between about, 50 to 150 nM, between about, 80-150 nM, for example, 80, 85, 90, 95,100, 105, 110 nM: between about, 15-100, between about, 20-40 nm, for example, 20, 25, 30, 35, 40, 45 nM; and an ALK5 inhibitor, such as RepSox, at a concentration of about 2-50 μM, preferably, 5-25 μM more preferably, 10-20 μM.

27. The method of any one of paragraphs 1-26, wherein the cell culture medium further comprises a cyclic AMP activator such as Forskolin at a concentration of about 2-50 μM, preferably, 5-25 μM more preferably, 10-20 μm μM).

28. The method of any one of paragraphs 1-27, wherein the cell culture medium further comprises an inhibitor of sonic hedgehog signaling such as SANTI at a concentration of about 0.05-2 μM, preferably, 0.1 to 1, and more preferably, 0.2-0.75 μM for example, 0.2, 0.25, 0.3, 0.35 μM.

28. The method of any one of paragraphs 1-26, wherein the cell culture medium comprises an ALK5 inhibitor, such as RepSox at a concentration of about 0.2-5 μM, preferably, 0.5-2.5 μM, more preferably, 1-2 μM.

29. The method of any one of paragraphs 1-28, wherein the cell culture medium comprises a notch inhibitor such as γ-Secretase Inhibitor XX at a concentration of about 20-500 nM, preferably, 50 to 150 nM, more preferably, 80-150 nM, for example, 80, 85, 90, 95,100, 105, 110 nM; an FGF inhibitor such as PD173074 at a concentration of about 0.02-0.5 μM, preferably, 0.05-0.25, more preferably, 0.08-0.15 μM, for example, 0.08, 0.09, 0.1, 0.15 μM, us in preferred embodiments, only for days 3-6).

30. The method of any one of paragraphs 1-28, wherein the cell culture medium comprises a NEUROD1 inducer, with ISX-9 at a concentration of about 2-50 μM, preferably, 5-25 μM more preferably, 8-15 μM, for example 8, 8, 10, 11, 12, 13, 14 or 15 μM, a GPER agonist, such as G-1 at a concentration of about 0.2-5 μM, preferably, 0.5-2.5 μM, more preferably, 1-2 μM, a histone methyltransferase inhibitor, for example, Deazaneplanocin-A used at a concentration of about 0.2-5 M, preferably, 0.5-2.5 μM, more preferably, 1-2 μM, an aurora kinase Inhibitor, with [0.1-2 mM, preferably, 0.1 to 1 mM, and more preferably, 0.2-0.75 mM for example, 0.2, 0.25, 0.3, 0.35 μM: an aurora kinase Inhibitor, for example, ZM447439 used at concentrations of about 0.1-2 mM, preferably, 0.1 to 1 mM, and more preferably, 0.2-0.75 mM for example, 0.2, 0.25, 0.3, 0.35 μM: and a pan-ErbB inhibitor (only used to supplement medium used for days 7-12), for example, CI-1033 used at a concentration of about 0.2-5 μM, preferably, 0.5-2.5 μM, more preferably, 1-2 μM.

31. A cell population comprising cells of pancreatic beta lineage obtained by the method according to any one of claims 1-20.

32. The cell population of paragraph 31, comprising NKX6.1+/INS+cells.

33. The cell population of claim 32, comprising INS+/GCG+ bi-hormonal cells, wherein the NKX6.1+/INS+ cells comprise at least about 50% of the total cell population.

33. The cell population of any one of claims 31-32, comprising least about %, 60%, 65%, 70%, 75%, 80%, 85%, 90%, 95%, or 99% NKX6.1+/INS+ cells.

34. The cell population of any one of claims 31-33, further comprising one or more cryoprotctants/cyroprotectant additives selected from the group consisting of dimethylsulfoxide (DMSO), thylene glycol (EG). antioxidants such as taurine, Metformin, and gamma amino butyric acid (GABA).

35. The cell population of any one of claims 31-33, in cell culture medium comprising one or more compounds selected from the group consisting of (i) betacellulin: (ii) a NEUROD1 inducer; (iii) a G protein-coupled estrogen receptor 1 (GPER) agonist: (iv) a histone methyltransferase inhibitor: (v) an aurora kinase inhibitor: (vi) a ROCK inhibitor; and (vii) a pan-ErbB inhibitor.

36. The cell population of claim 35, comprising one or more compounds selected from the group consisting of betacellulin, ISX-9, G-1, Deazaneplanocin-A, ZM447439, and H1152.

The invention is further described by way of the figures and data presented herein, and by way of examples.

### EXAMPLES

Material and methods
Mice

NOD-scid IL2 gamma null (NSG) mice were from The Jackson Laboratory. Mice were housed in 12-hr light/12-hr dark cycle. All procedures related to animals were performed in accordance with the ethical guidelines of the Salk Institute for Biological Studies. Animal protocols were reviewed and approved by the Salk Institute Institutional Animal Care and Use Committee (IACUC) before any experiments were performed. 6-to-8-week-old male NSG mice were used for surgery.
hESC Differentiation

hESCs were maintained as feeder-free in mTeSR™1 (STEMCELL Technologies Inc.) according to the manufacturer's instructions. Before differentiation, adherent hESCs (at ˜90% confluence) were rinsed with PBS and then incubated with Accutase (Millipore) for 6-8 min at 37° C. Dissociated single cells were rinsed twice with DMEM/F12 and spun at 300 g for 3 min. The resulting cells were re-suspended in mTeSR™1 which was supplied with 10μM Y27632 (Sigma-Aldrich), and seeded on 1:30 diluted Matrigel (BD Biosciences)-coated dishes at a density of 55,000 cells/cm². The next day, the medium was exchanged for mTeSR™MI and maintained for one more day prior to differentiation initiation.

Stage 1: Definitive Endoderm (3 days). After PBS rinse, undifferentiated hESCs were exposed to differentiation medium as following: day 1: MS12 medium supplied with 3 μM chir99021 and 115 ng/ml Activin-A (ActA, Peprotech); day 2: MS12 medium with 0.3 μM chir99021 and 110 ng/ml Activin-A: day 3: MS12 medium with 100 ng/ml Activin-A. Each 800 ml MS12 medium was made with 744 ml MCDB131 medium (ThermoFisher), 3.2 ml 45% glucose (Sigma), 20 ml 20% fat-acid free BSA, and 16 ml 7.5% sodium bicarbonate (Gibco).

Stage 2: Posterior Foregut (3 days): after PBS rinse, cells are exposed to MS12 medium with KGF (Peprotech, 50 ng/ml), B27 (Gibco, 100×), Vitamin C (Vc, 0.25 mM, sigma) and dorsomorphin (0.75 μM). Cells were fed with fresh medium daily.

Stage 3: Pancreatic endoderm (3 days): After PBS rinse, cells were exposed to DMEM with B27 (100×), retinoic acid (RA, 2 μM, Sigma), Noggin (100 ng/ml, Peprotech), SANTI (0.25 μM, sigma), Vitamin C (Vc, 0.25 mM, sigma), Pen/Strep (100×, Gibco) and Glutamax (100×, Gibco). Cells were fed with fresh medium every other day.

Stage 4: Pancreatic progenitor (3-4 days): After PBS rinse, cells were exposed to DMEM with B27 (100×), EGF (100 ng/ml, Peprotech)+Nicotinamide (NICO, 10 mM; Sigma)+Noggin (100ng/ml)+Vc (0.25 mM). Cells were fed with fresh medium every other day.

V-bottom plate based aggregation: stage-4 cells were rinsed with PBS and then incubated with Accutase (Millipore) for 10-15 min at 37° C. Dissociated single cells were rinsed twice with DMEM/F12 and spun at 300 g for 3 min. The resulting cells were re-suspended in an aggregation medium supplied with 10μM Y27632 (Sigma-Aldrich). Cell solution with 0.1-0.4 (according to experimental design) million cells was added into each well of V-bottom 96-well plate, followed by a spun at 300 g for 3 min. The plate was put into 37° C. incubator for 8-12 hours to form clusters. Aggregation medium was made of V4b-Medium+heparin (Sigma, H3149, 10 μg/ml)+ZnSO₄ (10 μM, sigma, Z0251)+LDN193189 (LDN, Tocris, 100 nM)+T3 (1 μM, Sigma, T6397)+RA (0.05 M)+SANTI (0.25 μM: Tocris)+GABA (1 mM, Sigma)+human EGF (100 ng/ml, Peprotech)+NICO (10 mM)+VC (0.25 mM). V4b-Medium (800 ml) was made of 360 ml MCDB 131 medium+180 ml F12 medium+180 ml KO-DMEM medium+3.9 ml Glucose (45%, Sigma)+80ml 20% FF-BSA+16 ml Sodium Bicarbonate (7.5%)+4 ml ITX+8 ml Pen/Strep+8ml Glutamax: all items were purchased from Thermo Fisher Scientific unless otherwise indicated.

Stage 5: PP-10C-treated pancreatic progenitor (4 days): Pancreatic progenitor clusters assembled in V-bottom 96-well plate were collected and rinsed twice using DF12 medium, and loaded on 6-well air-liquid interface trans-well (corning, 07200170). About 1.3 ml stage-5 medium was added for each well. Stage-5 medium was made of aggregation medium+TPB (PKC activator, 100 nM)+RepSox (10 μM, 374210, Thermo Fisher Scientific). Cells were fed with fresh medium every other day.

Stage 6: Endocrine progenitor (6 days). Clusters were rinsed in DF12 and transferred to a new trans-well. About 1.3 ml Stage-6 medium was added for each well. Cells were fed with fresh medium every other day. Stage-6 medium was made of B26-medium+Forskolin (10 μM)+T3 (1 μM). B26-medium=DMEM+B27 (100×), +LDN (500 nM)+TPB (30 nM)+RepSox (1 μM)+KGF (25 ng/ml)+SANTI (0.25 μM)+RA (0.05 μM)+PS (100×).

Stage 7: Immature βcells (12 days): Clusters were rinsed in DF12 and transferred to new trans-well. About 1.3 ml Stage-7 medium was added for each well. Cells were fed with fresh medium every other day. Stage-7 medium was made of 4#-medium, supplemented with (γ-Secretase Inhibitor XX (GSIXX), 100 nM, only for days 1-6)+RA (0.05 μM, only for days 1-6)+HGF (50 ng/ml, only for days 1-6)+IGF1 (50 ng/ml, only for days 1-6)+FGF inhibitor PD173074 (0.1 μM, only for days 3-6)). 4#-medium=V4b-medium+ZnSO₄ (10μM)+Heparin (10 mg/ml)+LDN (100 nM)+T3 (1μM)+RepSox (10 μM).

Stage 8: Functional βcells (12 days): Clusters were rinsed in DF12 and transferred to a new trans-well. About 1.3 ml Stage-8 medium was added for each well. Cells were fed with fresh medium every other day. Stage-8 medium was made of M18#-medium, supplemented with 7 chemicals/factors (termed as Fβ-7C, including betacellulin (BTC, 10 ng/ml), ISX-9 (a NEUROD1 inducer, 10 μM), G-1(a GPER agonist, 1 μM), Deazaneplanocin-A(Deza, a histone methyltransferase inhibitor, 1 μM), ZM447439 (ZM, an aurora kinase Inhibitor, 2.5 μM), H1152 (a ROCK inhibitor, 10 μM: only used for days 1-6), CI-1033 (a pan-ErbB inhibitor, 1 μM, only used for days 7-12). M18 medium (5.58 mM glucose) =MCD131 (Thermofisher, 340 ml for each 800 ml total medium)+F-12 (Thermofisher, 170 ml for each 800 ml total medium)+DMEM-no Glucose (Thermofisher, 170 ml for each 800 ml total medium)+fatty acid free BSA (final 2%, EMD Millipore, 126575)+7.5% Sodium Bicarbonate (12.8 ml for total 800 ml medium)+RPMI 1640 Vitamins Solution (100×, SIGMA-ALDRICH, R7256)+ITX (200×)+sodium pyruvate (200×)+NEAA (200×)+PS (100×)+Glutamax (100×)+45% glucose (Sigma, 2500×)+Heparin (10 mg/ml)+Lipid (2000×, Thermo Fisher Scientific, 11905031)+Trace Elements A (2000×, Corning, MT99182CI)+Trace Elements B (2000×, Corning, MT99175CI).

### Immunofluorescence and Imaging for Cell Culture

Cell culture staining: cell cultures were washed twice in PBS and fixed with 4% paraformaldehyde at room temperature for 15 minutes. Fixed cells were blocked in PBST (PBS containing 0.2% Triton100 and 0.5% normal donkey serum (Jackson Immuno Research Laboratories)) at room temperature for 1 hour. Primary and secondary antibodies were diluted in PBST. Cells were incubated with primary antibodies at 4° C. overnight, followed by three rinses and incubation with secondary antibodies at room temperature for 1 hour. The stained cells were rinsed with PBS and then incubated with DAPI (Sigma-Aldrich) for 2 minutes to stain the nuclei. Cells were then washed three times by PBS prior to imaging. TUNEL assays for detecting the in situ cell death were performed according to the manufactured manual (Roche). For confocal imaging, cells were usually mounted with a mounting medium and covered with a cover glass.

### Tissue Sectioning and Immunohistochemistry

Cell grafts were fixed with 4% paraformaldehyde at 4° C. for 2 hours, followed by three PBS washes at 4° C. (which lasted a few seconds, 10 min and 2 h). The cell grafts were then incubated in 30% (w/vol) sucrose solution at 4° C. overnight. The tissues were embedded in Optimal Cutting Temperature Compound (O.C.T) (Tissue-Tek), frozen in liquid nitrogen and sectioned at 7 μm using a Cryostat (Leica). Section staining was performed by using the same procedure as in “Cell culture staining” without the fixation step.

### Flow Cytometry and Cell Sorting

Single-cell suspension from cell cultures: Differentiated hESC cultures were rinsed with PBS and then incubated with 0.25% trypsin-EDTA (Life Technologies) at 37° C. for 1-3 minutes. The trypsin was neutralized with MEF medium. The dissociated cells were rinsed twice in PBS or DMEM/F12 medium for further analysis. For intracellular antibody staining, single cells were fixed in 200 μL of BD Cytofix/Cytoperm Buffer (BD Biosciences) at 4° C. for 20 minutes followed by three washes in BD Perm/Wash Buffer. Fixed cells were incubated in 150 μL of primary antibody buffer at 4° C. for 1 hour, followed by 30 minutes in a secondary antibody (if there is a secondary antibody) buffer after being rinsed twice in Perm/Wash Buffer. Stained cells were washed twice in Perm/Wash Buffer prior to analyses. The acquired data were analyzed by FlowJo.

Antibody Used in this Study

Primary Antibody used for Immunofluorescence and Immunohistochemistry including: NKX6.1 (DSHB, F55A12-c, 300×), PDX1(R&D, AF2419, 300×), INS-APC (Cell signaling, C27C9, 80×), NKX6.1-PE (BD, #563023, 40×), Human C-peptide (Abcam, Ab14181, 100×), Glucagon (Cell Signaling, 2760S, 400×), Glucagon (Abcam, ab82270, 100×), Insulin (Abcam, ab7842, 100×), GFP (Abcam, ab6673, 300×), NEUROD1 (R&D, AF2746, 200×), NEUROD1-APC (BD, 563000, 50×). Secondary antibodies were: Donkey anti-mouse Alexa Fluor 488 (Jackson Lab, 715-545-151), Donkey anti-Goat Alexa Fluor 488 (Jackson Lab, 705-545-147), Donkey anti-Rabbit Alexa Fluor 488 (Jackson Lab, 711-545-152), Donkey anti-Guinea Pig Alexa Fluor 488 (Jackson Lab, 706-545-148), Donkey anti-mouse Alexa Fluor 550 (Invitrogen, SA5-10167), Donkey anti-rat Alexa Fluor 555 (Abcam, ab150154), Donkey anti-Rabbit Alexa Fluor Cy3 (Jackson Lab, 711-165-152), Donkey anti-rat Alexa Fluor 647 (Abcam, ab150155), Donkey anti-Goat Alexa Fluor 647 (Jackson Lab, 705-605-147), Donkey anti-Guinea Pig Alexa Fluor 647 (Jackson Lab, 706-605-148), Donkey anti-mouse Alexa Fluor 647 (Jackson Lab, 715-605-151), Donkey anti-Guinea Pig FITC (Jackson Lab, 706-095-148), Donkey anti-Mouse Cy5 (Jackson Lab, 715-175-151), Donkey anti-Mouse Alexa Fluor 647 (AF647) (Jackson Lab, 715-605-151), and Donkey anti-Rabbit Alexa Fluor 647 (Jackson Lab, 711-605-152). All secondary were prepared following the manufacturers' instruction and used as 300-500×. Image acquisition was performed using a Zeiss LSM 710 confocal microscope. Images were processed using Fiji (ImageJ, v2.0.0) or Zen (Zeiss).

### Gene-targeting

A LoxP-flanked puromycin (Puro) selection, a 2A-NLS-GFP was knocked-in and fused in frame to the end of the endogenous NKX6. 1 coding sequence in H1 hESCs. The Puro cassette was later removed by adding

TAT-CRE protein. The cells showed nucleus-localized GFP expression once the endogenous NKX6.1 gene was activated. Targeting site of guide RNA for NKX6. 1 locus by CRISPR-Cas9: TGCGGCGGGCGGCGGCGTTC (SEQ ID NO:1). For targeting, a 1.3 Kb left homologous arm, and a 2.0 Kb right homologous arm were used.

### Static Glucose-stimulated Insulin Secretion

For static glucose-stimulated insulin secretion assays, stage-8 cells (usually 1-2 clusters, equivalent to ˜0.2-0.4 million cells in total), or 15 human islets were rinsed twice with Krebs buffer (129 mM NaCl, 4.8 mM KCl, 2.5 mM CaCl₂, 1.2 mM MgSO₂, 1 mM Na₂HPO₄, 1.2 mM KH₂PO₄, 5 mM NaHCO₃, 10 mM HEPES, 0.1% BSA, in deionized water and sterile filtered) and then pre-incubated in Krebs buffer for 60 mins. Cells were then incubated in Krebs buffer containing 3.3 mM glucose for 60mins. The cells were then transferred to a new plate containing Krebs buffer with 16.7 mM glucose (or other reagents) for 60 mins. Supernatant samples were collected after each incubation period and frozen for ELISA analysis. ELISA kits include human C-peptide ELISA (#10-1141-01; Mercodia).

### Total Insulin Content Measurement.

Human islets, stage-7 and stage-8 cells were incubated in Tris-EDTA (pH 7.4, on ice) and were sonicated to disrupt all cell membranes. After brief centrifugation, an aliquot of the lysed cell suspension (containing hormone) was measured using the insulin ELISA kits (#10-1113-01; Mercodia).

### In Vivo Animal Experiments

Transplantation assay was conducted as previously described (Cell Stem Cell. 2013 13(2):230-6). STZ (Intraperitoneal injection (I.P.), 35 mg/kg STZ daily for 4 days to create moderate diabetes). Mice were considered to be diabetic when blood glucose measurements were above 250 mg/dl for four consecutive days. About 1.6 million cells (equals about 8 clusters, each has ˜0.2 million cells) were transplanted under kidney capsule for each diabetic mouse. “No treatment” was acted as a sham surgery. For in vivo glucose-stimulated C-peptide secretion assay, human C-peptide levels were measured after overnight fasting and 60 min following an i.p. glucose bolus (2g glucose/kg body weight (2g/kg: 30% solution)) at 5 weeks post cell transplantations.

### Statistical Analyses

Before experiments, the sample size was not predetermined using any statistical methods. All values were expressed as the mean±SEM or mean±SD as indicated. Statistical analysis was performed using the Prism software (v7 or v8, GraphPad). Graphs were generated using Prism (Graphpad). P-values were determined by ANOVA or t-tests.

### Determining Conditions for Stage-5

To identify the conditions for the stage-5, the cells from the stage-4 were dissociated and aggregated (day 1) and cultured (days 2, 3, 4) on air-liquid surfaces using the 21 different conditions as listed in Table 1. The clusters were then fixed and sectioned for staining. Note: all the factors listed were used for the entire 4 days unless otherwise indicated.

TABLE 1
Conditions used to treat stage-4 cells.
Conditions = V4b-mdium + RA(0.05 μM) + SANT1(0.25 μM) + LND(0.1 μM) +
T3(1 μM) + ZnSO₄ + heparin(10 mg/ml) + TPB(0.1 μM) +
Y27632(used for only day 1(aggregation day)) + factors listed below

| | | | | | |
|---|---|---|---|---|---|
| 1 | GABA(1 mM) | | | | |
| 2 | GABA(1 mM) | EGF(50 ng/ml) | KGF(20 ng/ml) | | |
| 3 | | | | | |
| 4 | GABA(1 mM) | EGF(10 ng/ml) | KGF(20 ng/ml) | | repsox(10 μM), |
| | | | | | only for days |
| | | | | | 2, 3, 4 |
| 5 | GABA(1 mM) | | KGF(20 ng/ml) | | repsox(10 μM), |
| | | | | | only for days |
| | | | | | 2, 3, 4 |
| 6 | GABA(1 mM) | | | | repsox(10 μM), |
| | | | | | only for days |
| | | | | | 2, 3, 4 |
| 7 | | EGF(100 | NiCO(10 mM) | DBZ(1 μM) | repsox(10 μM), |
| | | ng/ml) | | | only for days |
| | | | | | 2, 3, 4 |
| 8 | GABA(1 mM) | EGF(100 | NiCO(10 mM) | DBZ(1 μM) | repsox(10 μM), |
| | | ng/ml) | | | only for days |
| | | | | | 2, 3, 4 |
| 9 | GABA(1 mM) | | | | repsox(10 μM), |
| | | | | | only for days |
| | | | | | 2, 3, 4 |
| 10 | | | | | repsox(10 μM), |
| | | | | | only for days |
| | | | | | 2, 3, 4 |
| 11 | GABA(1 mM) | | KGF(5 ng/ml) | | repsox(10 μM), |
| | | | | | only for days |
| | | | | | 2, 3, 4 |
| 12 | | | | | repsox(10 μM), |
| | | | | | only for days |
| | | | | | 2, 3, 4 |
| 13 | GABA(1 mM) | EGF(100 | NiCO(10 mM) | | repsox(10 μM), |
| | | ng/ml) | | | only for days |
| | | | | | 2, 3, 4 |
| 14 | | EGF(10 ng/ml) | | | repsox(10 μM), |
| | | | | | only for days |
| | | | | | 2, 3, 4 |
| 15 | GABA(1 mM) | EGF(10 ng/ml) | | | repsox(10 μM), |
| | | | | | only for days |
| | | | | | 2, 3, 4 |
| 16 | GABA(1 mM) | EGF(10 ng/ml) | | DBZ(1 μM) | repsox(10 μM), |
| | | | | | only for days |
| | | | | | 2, 3, 4 |
| 17 | GABA(1 mM) | EGF(10 ng/ml) | | DBZ(1 μM) | repsox(10 μM), |
| | | | | | only for days |
| | | | | | 2, 3, 4 |
| 18 | GABA(1 mM) | EGF(20 ng/ml) | NiCO(10 mM) | DBZ(1 μM) | repsox(10 μM), |
| | | | | | only for days |
| | | | | | 2, 3, 4 |
| 19 | GABA(1 mM) | EGF(50 ng/ml) | KGF(20 ng/ml) | | |
| 20 | | EGF(10 ng/ml) | | DBZ(1 μM) | repsox(10 μM), |
| | | | | | only for days |
| | | | | | 2, 3, 4 |
| 21 | | EGF(10 ng/ml) | | | repsox(10 μM), |
| | | | | | only for days |
| | | | | | 2, 3, 4 |

### Determining Conditions for Stage-6

To identify the conditions for the stage-6, the stage-5 clusters were treated with S6-Basic-condition supplemented with the extra factors as listed 5 in Table 2 for 6 days, followed by treated with later stage medium (made of 4#-medium, supplemented with (γ-Secretase Inhibitor XX (GSIXX), 100 nM, only for days 1-6)+RA(0.05 μm, only for days 1-6)+HGF(50 ng/ml, only for days 1-6)+IGF1(50 ng/ml, only for days 1-6)+FGF inhibitor PD173074 (0.1 μM, only for days 3-6)+BTC(10 ng/ml). 4#-medium=V4b-10 medium+ZnSO₄(10 mM)+Heparin(10 mg/ml)+LDN(100 nM)+T3(1 μM)+Repsox(10 μM)). In the end the clusters were dissociated into single cells for cell cytometry analysis for the expression of INS and NKX6.1.

TABLE 2
Conditions used to treat stage-5 clusters.
S6-Basic-condition = DMEM + B27 + LDN(0.5 μM) +
TPB(30 nM) + KGF (25 ng/ml)

| ID # | SANT1 | RA | FSK | T3 | Rep | |
|---|---|---|---|---|---|---|
| 2 | 0.25 μM | 0.05 μM | 10 μM | | 1 μM | |
| 3 | 0.25 μM | 0.05 μM | | 1 μM | | |
| 4 | 0.25 μM | 0.05 μM | 10 μM | 1 μM | 1 μM | BTC(10 ng/ml) |
| 5 | 0.25 μM | 0.05 μM | 30 μM | 1 μM | | |
| 6 | 0.25 μM | 0.05 μM | 10 μM | 1 μM | 1 μM | |
| 7 | 0.25 μM | | 10 μM | 1 μM | 1 μM | |
| 8 | 0.25 μM | 0.05 μM | 20 μM | 1 μM | | |
| 9 | 0.25 μM | 0.05 μM | 10 μM | 1 μM | 1 μM | extra |
| | | | | | | KGF(25 ng/ml) |
| 10 | 0.25 μM | 0.05 μM | 10 μM | 1 μM | 1 μM | EGF(10 ng/ml) |
| 11 | | 0.05 μM | 10 μM | 1 μM | 1 μM | |
| 12 | 0.25 μM | 0.05 μM | 10 μM | 1 μM | 1 μM | EGF(50 ng/ml) |
| 13 | 0.25 μM | 0.05 μM | 10 μM | 1 μM | 20 μM | |
| 14 | 0.25 μM | | | 1 μM | 1 μM | |
| 15 | 0.25 μM | | 10 μM | 1 μM | | |
| 16 | 0.25 μM | 0.05 μM | 10 μM | 1 μM | 10 μM | |

### Results

### Efficient Generation of Pancreatic Progenitor 3D Clusters

The generation of 3D clusters of pancreatic progenitors (PPs) is considered a necessary first step in producing β cells⁷. The protocol disclosed in Rezania et al. (Nature Biotechnology, 32:1121-1131 (2014)), hereafter, referred to as the R-protocol) was initially followed, as this protocol has been adopted by several other groups^{16, 18}. H1 hESCs were induced to form PPs (marked by PDX1+/NKX6.1+) in a step-wise process and then assembled into 3D clusters according to the R-protocol. However, this results in PP production with only moderate efficiency (˜30%). Moreover, the PP aggregates cannot form solidly, and constant cell detachments are observed. Therefore, uniform control of cluster size is difficult. Consequently, subjecting these PP 3D clusters to further differentiation yields only ˜8% to 25% of NKX6.1+/INS+ βcells. Other groups using the same procedures also reported similar β-cell generation efficiencies^{16, 18}.

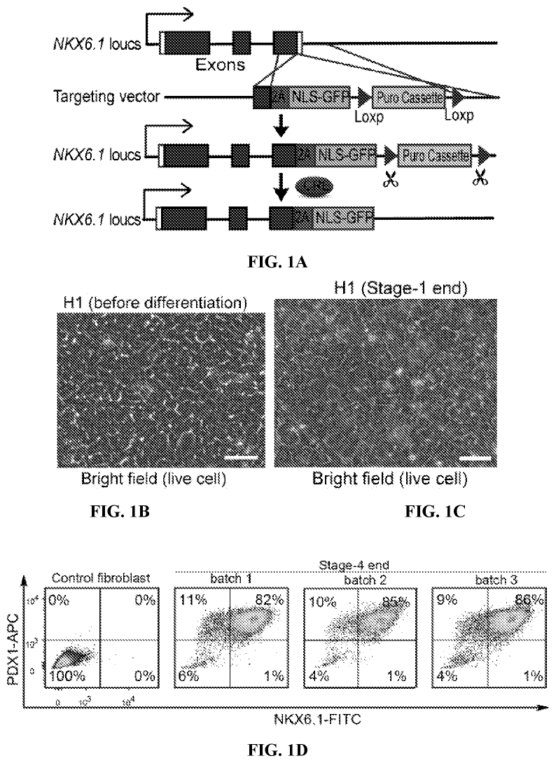

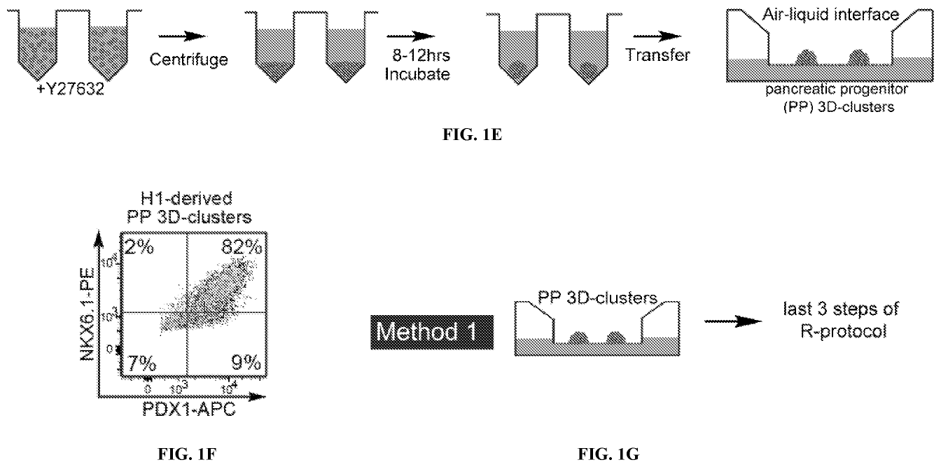

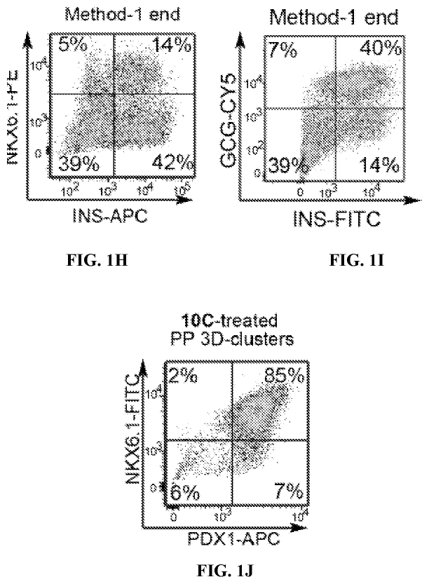

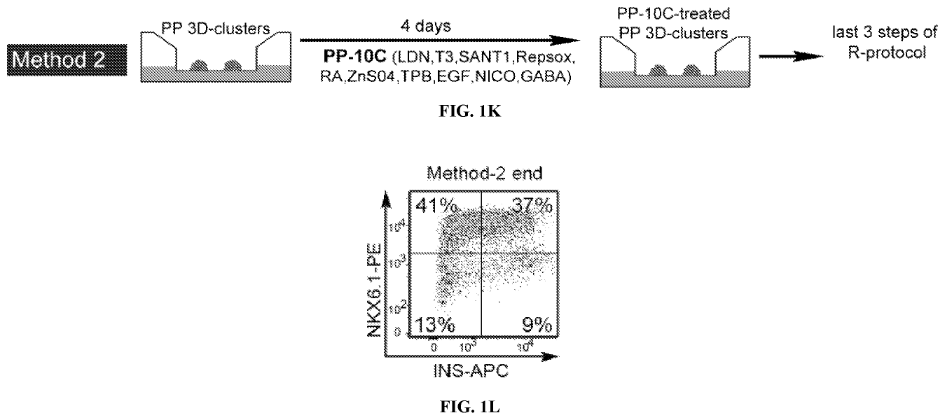

Efficient generation of PP 3D-clusters is responsible for generating functional βcells. An hPSC reporter line with NKX6.1-NLS-GFP has been generated by homologous recombination (FIG. 1A and data not shown), which aids in the study of functional β cell production protocols. Sequences encoding GFP and a nuclear localization signal (NLS) are inserted in the 3′ end of the endogenous NKX6. I gene in H1 hESCs. Restricting GFP to the nucleus increases local GFP fluorescence, enhancing the sensitivity so that live NKX6.1-GFP⁺ cells can be readily distinguished from NKX6.1-GFP⁻ cells. Using this reporter cell line, a new PP differentiation method has been developed. The chemically defined protocol (combination of stage 1, 2, 3 and 4, disclosed herein) produces a monolayer of NKX6.1+/PDX1+ PPs with an efficiency of 84%+2% (n=3) (FIG. 1D and data not shown). Of note, the dose of Activin-A at stage 1 is important, and a decreasing gradient over the 3-day treatment results in most PPs at stage 4 (data not shown). In contrast, treating cells with constant low-dose or high-dose of Activin-A results in low NKX6.1+ cells at the PP stage (Table 3).

TABLE 3
Effect of Activin A on treated cells

| Activin-A | | | |
|---|---|---|---|
| (ng/ml) | | | PDX1- |
| day 1-day 2- | PDX1-positve | NKX6.1-positve | postive/NKX6.1- |
| day 3 | cells | cells | positve cells |
| Constant low- | ~50%-80% | ~15%-50% | ~15%-50% |
| dose of Activin-A | | | |
| (100-100-100) | | | |
| Decreasing | ~93% | ~80% | ~80% |
| gradient dose of | | | |
| Activin-A | | | |
| (115-110-100) | | | |
| Constant high- | ~90% | ~20%-50% | ~20%-50% |
| dose of Activin-A | | | |
| (115-115-115) | | | |

A high-dose Activin-A severely reduced cell proliferation and resulted in suboptimal cell density. On the other hand, constant low-dose Activin-A generated cell culture with appropriate cell density but resulting in high cell heterogeneity at stage 4 (data not shown). These observations demonstrate that a balance between differentiation and proliferation is important for the efficient generation of PPs.

Improved aggregation methods used to assemble these PPs into 3D clusters are also provided herein. To accomplish this, a V-bottom plate was used (FIG. 1E). Dissociated PP cells were added to V-bottom wells of a 96-well plate in an aggregation medium supplemented with the ROCK inhibitor Y-27632 (which increases the viability of dissociated cells). Approximately 0.2 million cells were pelleted in the bottom of the well by centrifugation. Compact 3D clusters were observed after overnight incubation, and the clusters were then transferred to the air-liquid interface for further differentiation. Using this method, compact 3D clusters of PPs were generated without significant loss of cells. These PP 3D clusters uniformly express high levels of NKX6.1-NLS-GFP, which is in sharp contrast to aggregates from undifferentiated hPSCs (data not shown). FACS analysis of the PP 3D clusters in three different batches of differentiation revealed 84%±2% (n=3) of PDX1+/NKX6.1+ cells in the whole population (FIG. 1D). To test the reproducibility of this new protocol, it was applied to original wild-type H1 cells without the reporter and a similar efficiency (81%±4%. n=3) in generating PPs and assembling them into 3D clusters with uniform sizes (FIG. 1F and data not shown), was obtained. Taken together, a new protocol for the efficient generation of 3D clusters of PPs from hPSCs has been successfully established.

### 10 Chemicals Poise Pancreatic Progenitors in 3D Clusters

Subsequent studies sought to differentiate H1 hESC-derived 3D PP clusters into β cells.
Method 1

The last three steps of the R-protocol were applied to differentiate 3D PP clusters (this combined protocol is referred to as Method 1). Despite the production of ˜56% INS+ cells, most of INS+ cells also expressed GCG, and only 14%±4% (n=3) of total cells are βcells (marked by NKX6.1+/INS+) (FIGS. 1G-1I). The percentage of NKX6.1− expressing cells (i.e., NKX6.1+ cells) drops from ˜80% to ˜19% after the last three stages of the R-protocol. The prevalence of INS+/GCG+/NKX6.1− cells show that the last three steps of the R-protocol mainly induce the PP clusters into INS+/GCG+ bi-hormonal cells, rather than INS+/NKX6.1+ βcells. Since it has been reported that most INS+/NKX6.1− cells expressed GCG¹⁷, and given the role of NKX6.1 for maintaining the functional state of β cells²¹, it was hypothesized that the premature loss of NKX6.1 expression and/or PPs identity led to the INS+/GCG+/NKX6.1− cell fate. Therefore, maintaining cells in the PP stage with compact 3D structures until they are fully committed may enhance their propensity to become β cells.

3D clusters of PPs were treated with different combinations of factors, screening for conditions that retain NKX6.1 expression in the PP state without compromising the compact state of the 3D structures. In many of the tested conditions, NKX6.1 expression dropped significantly just four days after the formation of PP-3D clusters (data not shown), demonstrating that NKX6.1 expression can be lost prematurely under undesirable conditions. Only condition #13 maintained NKX6.1 expression after 4 days of treatment (data not shown). Condition #13 contains 10 chemicals, namely LDN193189 (LDN, an inhibitor of BMP signaling), T3 (thyroid hormone), retinoic acid (RA), SANTI (an inhibitor of sonic hedgehog signaling), Repsox (an ALK5 inhibitor), ZnSO4, TPB (a PKC activator), EGF, Nicotinamide (a form of vitamin B3), and Gamma-aminobutyric acid (GABA, a neurotransmitter). Condition #13 is a novel combination that has not been used for treating pancreatic progenitors: this combination of 10 chemical/factors is referred to herein as “PP-10C”. Further characterization of PP-10C-treated PPs showed that 83%±5% (n=3) cells expressed both PDX1 (pancreatic and duodenal homeobox 1) and NKX6.1but not middle- or late-stage markers of endocrine cells, such as NGN3 or NEUROD1 (FIG. 1J and data not shown). The results of terminal deoxynucleotidyl transferase dUTP nick end labeling (TUNEL) staining (for detecting cell death) combined with staining for NKX6.1 and DAPI revealed very few dying/dead cells in cells treated with the PP-10C condition (data not shown). In contrast, more dying/dead cells have been observed in other conditions such as #4, #5, and #19. For example, necrotic cores (with a lot of pervading DAPI stain, dying/dead cells, and cell debris) were observed in some conditions (conditions #4, #5, and #19, data not shown). Together, this shows that PP-10C not only efficiently maintains the undifferentiated status of PPs but also retains the viability of all cells in 3D clusters.
Method 2

The potential for these PP-10C-treated PP clusters to differentiate into βcells, was tested. Subjecting PP-10C-treated PPs to the last 3 steps of the R-protocol (this entire experimental protocol is referred to herein as Method 2) produces 36%±6% (n=3) NKX6.1+/INS+ βcells (a ˜3-fold increase over Method 1) and only ˜9% INS+/NKX6.1− byproduct cells (a ˜5-fold decrease over Method 1) (FIGS. 1K and 1L). Thus, PP-10C treatment increases the potential of PPs in 3D clusters to differentiate into β cells, while reducing their potential to become unwanted INS+/GCG+byproduct cells.

### Identifying Chemical Combinations for Inducing β Cells

To improve βcell yield, a step-wise procedure for differentiating PP-10C-treated PPs into endocrine progenitors (EP), then into immature β (iβ) cells, and finally functional B (FB) cells was used (FIG. 2A). The last three steps of this stepwise procedure are referred to as stage 6, 7, and 8, respectively, as five previous stages are needed to differentiate hPSCs into PP-10C-treated PPs (FIG. 2A).
Method 3

To search for potentially unknown signaling pathways involved in these differentiation stages, a custom screening library containing more than one hundred chemicals/growth factors was generated, and this library included factors that can modulate (activate or inhibit) most of the known developmental and differentiation-related signaling pathways (Table 4). To this end. >2000 conditions were systematically screened using different combinations of chemicals/factors from the library.

TABLE 4
A custom library of growth factors and compounds
modulating most of the known development and
differentiation-related signaling pathways.

| | Function | |
|---|---|---|
| Signaling pathway/ | (inhibitor/ | Chemical/factor name |
| target | Activator) | (concentration tested) |
| WNT | Inhibitor | XAV939(0.1-5 μM) |
| WNT | Inhibitor | wnt-c59(0.01, 2 μM) |
| WNT | Activator | wnt3a (2, 20 ng/ml) |
| WNT | Inhibitor | IWR1-1-endo (2.5 μM) |
| WNT | Inhibitor | IWP2(0.1-2 μM) |
| WNT | Activator | CHIR99021(1, 3 μM) |
| Vitamin | Others | VC (0.25 mM) |
| Vitamin | Others | Nicotinamide (10 mM) |
| VEGF | Activator | VEGF (50 ng/ml) |
| TGFBeta | Activator | TGFBeta3(1-5 ng/μl) |
| TGFBeta | Activator | TGFBetal(1-5 ng/μl) |
| TGFBeta | Inhibitor | SB431542(2 μM) |
| TGFBeta | Inhibitor | Repsox (1, 2, 10 μM) |
| TGFBeta | Activator | human Activin A (2, 10, 50 |
| | | ng/ml) |
| TGFBeta | Inhibitor | A83-01 (0.5 μM) |
| TGF(BMP), AMPK | Inhibitor | Dorsomorphin (0.75 μM) |
| TGF(BMP) | Inhibitor | Noggin (200 ng/ml) |
| TGF(BMP) | Inhibitor | LDN193189 (0.1 μM) |
| TGF(BMP) | Activator | human BMP4 (20 ng/ml) |
| Ribosomal S6 kinase | Inhibitor | BRD7389 |
| (RSK) | | |
| RHO/ROCK | Inhibitor | Y27632 (10 μM) |
| RHO/ROCK | Inhibitor | H1152 (5 μM) |
| RHO/ROCK | Inhibitor | Fasudil (3 μM) |
| RA (retinoic acid) | Activator | RA (0.01, 1 μM) |
| RA (retinoic acid) | Inhibitor | LE135 (1 μM) |
| RA (retinoic acid) | Inhibitor | BMS195614 (1-2 μM) |
| RA (retinoic acid) | Activator | AM580 (0.05 μM) |
| RA (retinoic acid) | Inhibitor | AGN193109 (1 μM) |
| Pluripotency related | others | and minocycline |
| | | hydrochloride(2 μM) |
| Pluripotency related | others | (S)-(+)-dimethindene maleate |
| | | (DiM) (MiH)(2 μM) |
| PLK4 | Inhibitor | Centrinone B (30 nM) |
| PKC | Activator | TPB (0.2 μM) |
| PI3K | Inhibitor | Ly294002 (2, 20 μM) |
| PI3K | Activator | hμMan LIF (10 ng/ml) |
| PI3K | Activator | hμMan IGF2 (50 ng/ml) |
| PI3K | Activator | hμMan IGF1 (50 ng/ml) |
| PI3K | Activator | hIGF-II (50 ng/ml) |
| PDGF | Inhibitor | SU16F (2 μM) |
| PDGF | Inhibitor | JNJ10198409 (0.1 μM) |
| PDGF | Activator | human PDGFAB(20 ng/ml) |
| P70 | Inhibitor | LY2584702 (2 μM) |
| P38, Pluripotency | Inhibitor | SB202190 (10-20 μM) |
| related | | |
| P38, MAPK | Inhibitor | SB203580 (20 μM) |
| Others | Others | Zinc sulfate heptahydrate |
| Others | Others | Heparin (10 ug/ml) |
| Others | Others | GABA (1 mM) |
| Others | Others | dexamethasone |
| NOTCH-SRC | Inhibitor | PP2 (5 μM) |
| NOTCH | Activator | JAG-1 (100 ng/ml) |
| NOTCH | Inhibitor | GSiXX (0.1 μM) |
| Notch | others | DLl4 (250 ng/ml) |
| Notch | others | DLl1 (250 ng/ml) |
| Notch | Inhibitor | DAPT(1 μM) |
| mTOR | Inhibitor | rapamycin (5 nM) |
| Monoamine oxidase | Inhibitor | Tranylcypromine(Parnate) |
| | | (5 μM) |
| MEK | Inhibitor | PD0325901 (1 μM) |
| Lysophosphatidic acid | Activator | LPA (1-2 μM) |
| (LPA) | | |
| JNK, Protein synthesis | others | Anisomycin (5 μM) |
| JNK | Inhibitor | SP600125 (10-20 μM) |
| Hypoxia | others | CoCl2 (100-200 μM) |
| Hormone | Activator | T3 (0.1, 1 μM) |
| HGF | Activator | HGF (50 ng/ml) |
| Hedgehog/Smoothened | Inhibitor | sant1 (0.25 μM) |
| Hedgehog/Smoothened | Activator | Hh-Ag1.5 (0.2 μM) |
| Hedgehog/Smoothened | Inhibitor | GDC0449 (0.1-2 μM) |
| GPER | Activator | G-1 (1 μM) |
| GLP1 | Activator | Exendin-4 (50 ng/ml) |
| Gene activator | NeuroD1 inducer | ISX-9 (10 μM) |
| Gene activator | PDX1 inducer | BRD7552 (5 μM) |
| GDNF | Activator | hGDNF (50 ng/ml) |
| G9a/GLP | Inhibitor | BIX-01294 (1 μM) |
| FGF | Inhibitor | SU5402 (2 μM) |
| FGF | Inhibitor | PD173074 (0.1 μM) |
| FGF | Activator | human bFGF (1, 5, 20 ng/ml) |
| FGF | Activator | hFgf9 (20 ng/ml) |
| FGF | Activator | hFGF4 (50 ng/ml) |
| FGF | Activator | hFGF10 (50 ng/ml) |
| FGF | Activator | hFGF1 (50 ng/ml) |
| FGF | Activator | FGF7 (KGF, 5-20 ng/ml) |
| ER-beta | Activator | WAY-200070 (0.1, 1 μM) |
| ER-beta | Activator | AC-186 (0.5 μM) |
| ER-alpha | Activator | PPT (0.1 μM) |
| ER-alpha | Inhibitor | MPP dihydrochloride (2 μM) |
| Epigenetics | Inhibitor | VPA (0.5 mM) |
| Epigenetics | Inhibitor | TSA (0.02 μM) |
| Epigenetics | Inhibitor | RG108 (0.1-10 μM) |
| Epigenetics | Inhibitor | NaB (0.2 mM) |
| Epigenetics | Inhibitor | DEZA; Deazaneplanocin A |
| | | (1 μM) |
| Epigenetics | others | 5-Azacytidine (AZT, 5 μM) |
| EGFR and ErbB2 | Inhibitor | CI-1033 (1 μM) |
| EGF related | Activator | Betacellulin (20 ng/ml) |
| EGF | Activator | hEGF (10, 50, 1 00 ng/ml) |
| EGF | Inhibitor | Erlotinib (10 μM) |
| DOT1L | Inhibitor | SGC 0946 (2.5 μM) |
| DOT1L | Inhibitor | EPZ004777(5 μM) |
| cAMP, PKA | Activator | 8-Br-cAMP (0.1-0.3 mM) |
| cAMP | Activator | Forskolin (10 μM) |
| cAMP | Activator | dbcAMP (0.1-1 mM) |
| Calcium signaling | others | LONO (1 μM) |
| Calcium signaling | Activator | BayK8644 (2 μM) |
| AXL, RSK | Inhibitor | R428 (2 μM) |
| Autophagy | others | SMER28 (50 μM) |
| Aurora kinase | Inhibitor | ZM447439 (ZM, 0.5, 2.5 μM) |
| Aurora kinase | Inhibitor | AMG-900 (1 μM) |
| ATPase | Inhibitor | Blebbistatin (1-10 μM) |
| Anti-diabetes related | others | Pioglitazone HCl (10 μM) |

For stage 6, the classic endocrine progenitor markers, NKX6.1+/NEUROD1+, are initially used as the readout. Other conditions that efficiently generated NKX6.1+/NEUROD1+ cells from PP-10C-treated

PPs have been identified (FIG. 2B). However, NKX6.1+/NEUROD1+ cells generated by these conditions failed to efficiently become βcells following further differentiation (data not shown). This shows that the expression of a few marker genes characteristic of an intermediate stage is not necessarily a good indicator of their potential to differentiate into β cells.

Thus, a more stringent screen has been designed that uses late-stage markers (NKX6.1+/INS+ for this case) as the readout, referred to as “late-stage readout strategy” (FIGS. 2D-2E). Using this strategy, at stage 6 (endocrine progenitor stage), a combination of 8 chemicals/factors (termed EP-8C) has been identified that gives rise to the highest percentage of NKX6.1+/INS+ cells after further differentiation (FIGS. 2A, and 2D-2E). The EP-8C combination is forskolin (FSK, a cAMP pathway activator), Repsox (only 1 μM), LDN, TPB, KGF, SANTI, RA, and T3. Adding FSK and using a low concentration of Repsox is important for this differentiation process. Retrospective analysis shows that EP-8C also efficiently generates NKX6.1+/NEUROD1+ cells at stage 6 (data not shown).

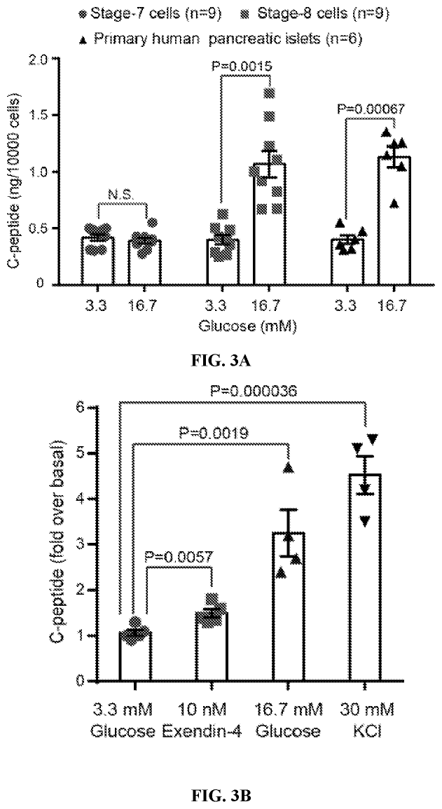

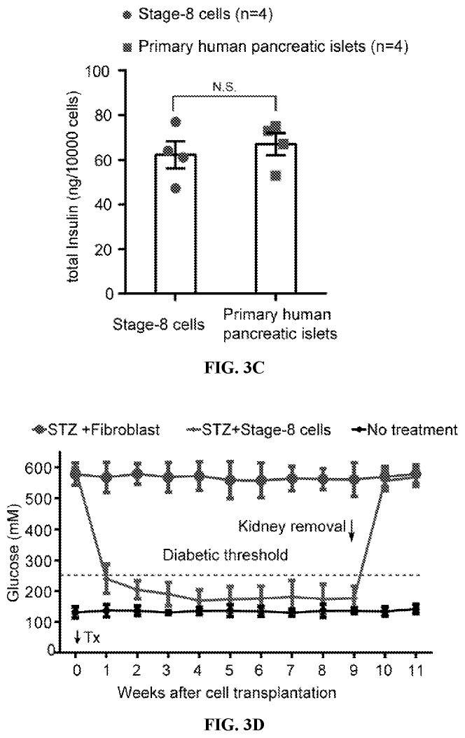

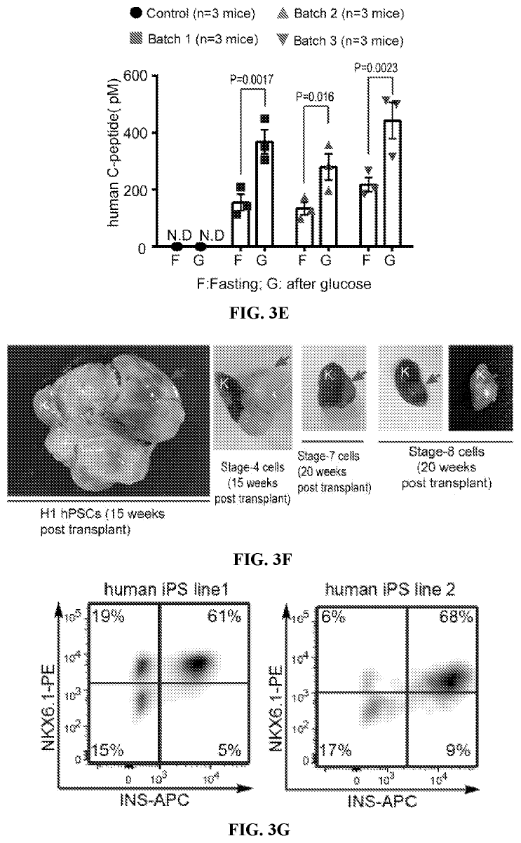

Stage 7 and 8 combinations of compounds that show improved results were also identified: the conditions identified are shown in FIG. 2A. Specifically, stage 7 (immature β cell stage) requires 9 chemicals/factors (termed as iβ-9C), namely LDN, T3, Repsox, ZnS04, GSIXX (a notch pathway inhibitor), RA, HGF, IGF1, and PD173074 (PD, an FGF pathway inhibitor)) (FIG. 2A and FIG. 2G). Stage 8 (functional β cell stage) requires 7 chemicals/factors (termed as Fβ-7C), namely betacellulin (BTC), ISX-9 (a NEUROD1 inducer), G-1 (a GPER agonist), Deazaneplanocin-A (Deza, a histone methyltransferase inhibitor), ZM447439 (ZM, an aurora kinase inhibitor), H1152 (a ROCK-II inhibitor), and CI-1033 (a pan-ErbB inhibitor) (FIGS. 2A, and 2F). Therefore, a novel 3-stage protocol has been established for inducing PP-10C-treated-PPs into βcells (FIG. 2A). This new experimental protocol is referred to as Method 3. EP-8C, iβ-9C, and Fβ-7C are all new combinations of chemicals/factors. Some of these chemicals/factors (including FSK for stage 6, and ISX9, G-1, Deza, ZM, and CI-1033 for the stage 8) have not been used previously, to differentiate hPSCs into β cells (FIG. 2A). Using Method 3, NKX6.1+/INS+ β cells were generated with an efficiency of up to 82% (75%±6%, n=5) (FIG. 2F and Table 5). For stage 8, each Fβ-7C component enhances the generation of β cells, as removing any component compromises production of NKX6.1+/INS+ βcells (FIG. 2F). In agreement with the data from flow cytometry, immunostaining sections of stage 8 cell aggregates reveal robust expression of NKX6.1 and INS, and only a few cells that are dual hormonal byproduct cells that expressed GCG and INS but not NKX6.1 (FIG. 2C and data not shown). In addition, C-peptide (a stoichiometric indicator of proinsulin processing) and PDX1 (a transcription factor expressed by mature βcells) were also robustly expressed in the stage 8 clusters (data not shown). A total of five cell lines (including H1, H1-NKX6.1-GFP; and three integration-free hiPSC lines (hiPSC1, hiPSC2 and hiPSC3) were tested using this protocol. More than 60% βcells were routinely generated from any of the cell lines (FIG. 3G and Table 5). In addition, this protocol is reproducible for the same cell line with low batch variations. These results demonstrate that this protocol is suitable for many ES and high-quality iPS cell lines.

TABLE 5
Generation of β cells from multiple hPSC lines in different tests.

| | Efficiency of β cell generations in different |
|---|---|
| Cell line | tests |
| H1 hESC | 78%; 82%; 67%; 75%; 74% |
| H1 hESC-NKX6.1- | 75%; 64%; 77% |
| GFP | |
| hiPSC1 | 61%; 57% |
| hiPSC2 | 68%; 60%; 63% |
| hiPSC3 | 63% |

βcells were Functional Both In Vitro and In Vivo

Finally, physiological tests on the stage 8 βcells from Method 3 were performed in vitro and in vivo. For examining glucose sensing activity was used; an in vitro glucose stimulated C-peptide secretion assay showed that the stage 8 cells respond to high-concentration (16.7 mM) glucose but not low-concentration (3.3 mM) glucose, and release human C-peptide (as normally seen from isolated human pancreatic islets) (FIG. 3A). The C-peptide levels induced by glucose from stage 8 cells are similar to primary human pancreatic islets. In contrast, immature stage 7 cells do not respond to high-concentration (16.7 mM) glucose (FIG. 3A). Stage 8 cells also respond to other secretagogues, including Exendin-4 and KCl (data not shown). In addition, the total insulin content in stage 8 cells was measured, and found to be ˜62 ng per 10,000 cells, a value comparable to that in primary human islets (FIG. 3C).

To functionally test the stage 8 cells in vivo, these cells were transplanted under the kidney capsule of streptozotocin (STZ)-induced diabetic NOD SCID gamma (NSG) mice. Diabetic mice that received these transplanted cells quickly recovered to normoglycemia (within ˜2 weeks), whereas diabetic mice that received fibroblasts as a control maintain hyperglycemia (FIG. 3D). The in vivo glucose stimulated C-peptide secretion assay also revealed robust secretion of human C-peptide in diabetic mice transplanted with the stage 8 cells. In these mice, about two times more C-peptide was detected after glucose stimulation compared to the fasting state (before glucose stimulus) (FIG. 3E). To test the safety of these β cells, cells from different differentiation stages (including, undifferentiated H1 hPSCs, stage-4, -7 and -8 cells) have been transplanted into normal NSG mice. As shown in FIG. 3F in contrast to the massive teratoma formed by H1 hPSCs after 15 weeks, the stage 8 and stage 7 cells do not form teratomas even after 20 weeks post-transplantation. Stage 4 cells form some cyst-like structures (FIG. 3F). Analysis of the stage 8 cell-derived engraftments showed robust expression of INS (data not shown). Collectively, the β cells generated using the protocol developed herein exhibit functionality both in vitro and in vivo.

### Discussion

Efficient generation of functional βcells from hPSCs (for the treatment of diabetes) has been a persistent challenge in the field of regenerative medicine. Current methods generate β cells with sub-optimal efficiency, and the effectiveness of these previously published protocols varies when applied to different cell lines. A robust chemical recipe for a highly efficient production of NKX6.1+/INS+ βcells from hPSCs is established herein (data not shown). This disclosed method incorporates several features: 1) a chemically defined protocol for the efficient generation of PPs, 2) an improved method for assembling PPs into 3D clusters, 3) a 10-chemical/factor cocktail (PP-10C) that maintains 3D-PPs status and enhances their potential to differentiate into βcells (rather than unwanted by-products such as GCG+/INS+ cells), and 4) a 3-step differentiation protocol (with combinations of signaling pathway regulators that have not been reported for each step) that efficiently converts PP-10C-treated 3D-PPs into functional β cells. As this method (when compared to other methods) significantly increases the efficiency of generating βcells from multiple cell lines with reduced unwanted cellular byproducts, it can promote both basic research and clinical translation of hPSC-derived β cells.

One principle concerning the differentiation of hPSC into a specific cell type of interest is that cells must be induced to transiently progress through several progenitor states. The importance of poising certain 3D-progenitor states during the differentiation process has been demonstrated. These steps are beneficial because the transient appearance of a progenitor state does not sufficiently entail the full acquisition of the cell state, and the premature loss of a progenitor state often leads to the generation of undesirable cellular byproducts in the downstream steps. The strategy of poising progenitor cells during the differentiation process disclosed herein, is applicable for generating a range of cell types from hPSCs.

Another useful strategy implemented in the screening process is the use of late-stage gene markers as the readout for the intermediate step (referred to as the late-stage readout-strategy). The resulting conditions using this strategy are generally more reliable and reproducible than those identified solely based on stage specific gene expression marker(s). This strategy can be adapted for generating other cell types from hPSCs. The results also reveal the importance of several signaling pathways modulators (e.g. FSK, a cAMP pathway activator, and CI-1033, a pan-ErbB inhibitor) in promoting the differentiation of hPSCs into β cells.

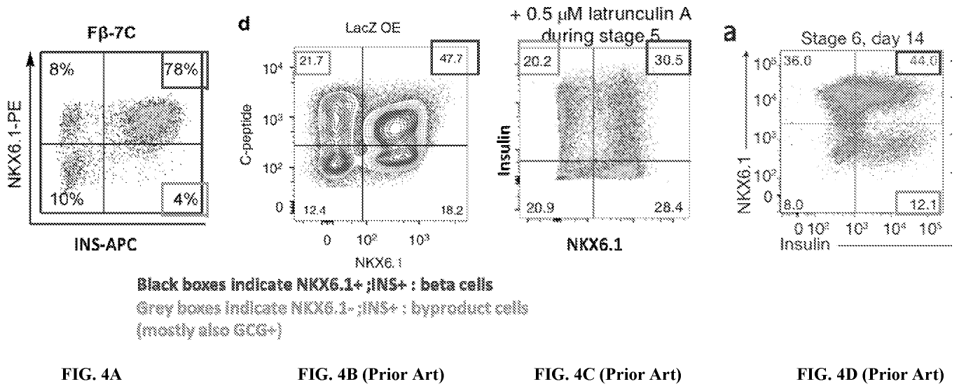

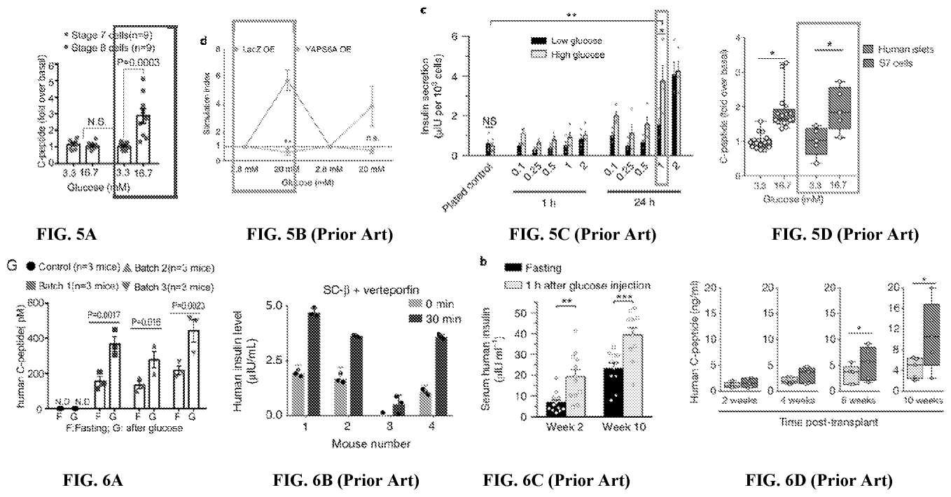

The efficiency of β cells formation, static glucose-stimulated insulin secretion (GSIS), and in vivo GSIS using the disclosed strategies are compared with those reported in the literature. The results are shown in FIGS. 4A-4D (βcell efficiency), FIGS. 5A-5D (static GSIS), and FIGS. 6A-6D (in vivo GSIS), and Tables 6 and 7.

TABLE 6
Comparison of static GSIS using the strategy developed
herein with reports in the literature.

| | Strategy developed | | | |
|---|---|---|---|---|
| | herein | Peterson | Hogrebe | Rezania |
| Induction | 3.3 mM vs | 2.8 mM vs | 2 mM vs | 3.3 mM vs |
| method | 16.7 mM | 20 mM | 20 mM | 16.7 mM |
| Fold | ~3 fold | ~5.5 fold | ~2.5 fold | ~2 fold |
| induction | | | | |

TABLE 7
Comparison of in vivo GSIS using the strategy developed
herein with reports in the literature.

| | Strategy developed | | | |
|---|---|---|---|---|
| | herein | Peterson | Hogrebe | Rezania |
| Fold induction | ~2 fold | ~2 fold | ~2 fold | ~2 fold |

### REFERENCES

1. Baeyens, et al. PHYSIOL REV 98, 1143-1167 (2018).
2. Ghazizadeh, et al. NAT COMMUN 8, 298 (2017).
3. Jiang, et al. CELL RES 17, 333-344 (2007).
4. Kroon, et al. NAT BIOTECHNOL 26, 443-452 (2008).
5. Kushner, et al. CELL STEM CELL 15, 535-536 (2014).
6. Pagliuca, W. et al. CELL 159, 428-439 (2014).
7. Rezania, et al. NAT BIOTECHNOL 32, 1121-1133 (2014).
8. Russ, et al. EMBO J 34, 1759-1772 (2015).
9. Vegas, et al. NAT MED 22, 306-311 (2016).
10. Yamashita-Sugahara, et al. SCI REP-UK 6 (2016).
11. Hogrebe, et al. NAT BIOTECHNOL 38, 460-470 (2020).
12. Nair, et al. NAT CELL BIOL 21, 263-274 (2019).
13. Zheng, et al. FRONT CELL NEUROSCI 12, 239 (2018).
14. Fujikawa, et al. AM J PATHOL 166, 1781-1791 (2005).
15. Hentze, et al. STEM CELL RES 2, 198-210 (2009).
16. Sui, et al. DIABETES 67, 26-35 (2018).
17. Veres, et al. NATURE 569, 368-373 (2019).
18. Petersen, et al. STEM CELL REP 9, 1246-1261 (2017).
19. Liu, et al. CELL RES 24, 1181-1200 (2014).
20. Nostro, et al. STEM CELL REP 4, 591-604 (2015).
21. Brandon, et al. Nkx6.1 CELL REPORTS 4, 1262-1275 (2013).
22. Slaymaker, et al. SCIENCE 351, 84-88 (2016).
23. Takahashi, et al. SCIENCE 356, 503-508 (2017).
24. Maruyama, et al. NAT BIOTECHNOL 33, 538-542 (2015).
25. Gouzi, et al. Dev Dyn 240, 589-604 (2011).
26. Rukstalis, et al. GENE EXPR PATTERNS 7, 471-479 (2007).
27. Cole, et al. J ENDOCRINOL 203, 19-31 (2009).
28. Xue, et al. INT J ONCOL 56, 1129-1139 (2020).
29. Willems, et al. CELL DIV 13, 7 (2018).
27. Myster, et al. Curr Biol, 2000. 10(8): p. R302-4.
30. Dioum, et al. PNAS 108, 20713-20718 (2011).
31. Saber, et al. ENDOCRINOLOGY 159, 1827-1841 (2018).

1. A method of generating cells of pancreatic beta lineage from pluripotent stem cells comprising:
(a) culturing the pluripotent stem cells in a cell culture medium comprising one or more compounds with biological activities selected from the group consisting of: (i) a Wnt signaling activator. and (ii) a BMP signaling inhibitor to obtain a population of definitive endoderm (DE) cells;
(b) culturing the DE cells to cell culture medium comprising one or more compounds selected from the group consisting of: i) a growth factor, (ii) a TGFβ receptor (BMP) inhibitor and (iii) vitamin C to obtain primitive gut tube (PGT) cells;
(c) culturing the PGT cells in cell culture medium comprising one or more compounds selected from the group consisting of: (i) a growth factor, (ii) a transforming growth factor (TGF)β receptor (BMP) inhibitor, (iii) vitamin C; (iv) a retinoic acid signaling activator, and (v) an inhibitor of sonic hedgehog signaling; to form posterior gut (PG) cells;
(d) culturing PG cells in cell culture medium comprising one or more compounds selected from the group consisting of: (i) a growth factor, (ii) a TGFβ receptor (BMP) inhibitor (iii) vitamin C and (iv) nicotinamide to obtain pancreatic progenitor (PP) cells;
(e) culturing the PP cells in aggregation medium to form PP-3D clusters,
wherein the aggregation medium is cell culture medium comprising one or more compounds selected from the group consisting of: i) a growth factor; (ii) a TGFβ receptor (BMP) inhibitor; (iii) vitamin C; (iv) a retinoic acid signaling activator; (v) an inhibitor of sonic hedgehog signaling; (vi) nicotinamide; (vii) thyroid hormone; (viii) γ-amino butyric acid (GABA and (ix) ZnSO4; and
wherein the aggregation medium comprises a RHO/ROCK (Rho-associated, coiled-coil containing protein kinase) inhibitor;
(f) culturing the PP-3D clusters in aggregation medium comprising one or more compounds selected from the group consisting of: (i) a PKC activator and (ii) an ALK5 (TGFβ receptor 1) inhibitor, to form primed PP cells;
(g) culturing the primed PP cells in cell culture medium comprising one or more compounds selected from the group consisting of: (i) a growth factor; (ii) a TGFβ receptor (BMP) inhibitor; (iii) a cyclic AMP activator; (iv) a retinoic acid signaling activator; (v) an inhibitor of sonic hedgehog signaling; (vi) an ALK5 inhibitor; (vii) thyroid hormone and (viii) a PKC activator, to form NKX6.1+/NEUROD1⁺ endocrine progenitor (EP) cells;
(h) culturing the EP cells in cell culture medium supplemented with one or more compounds selected from the group consisting of (i) a growth factor; (ii) a TGFβ receptor (BMP) inhibitor; (iii) an inhibitor of notch signaling; (iv) a retinoic acid signaling activator; (v) an ALK5 inhibitor; (vi) thyroid hormone; (vii) a Fibroblast growth factor (FGF) inhibitor and (ix) ZnSO₄, to form immature β cells (IBC); and
(i) culturing the IBC in cell culture medium comprising one or more compounds selected from the group consisting of: (i) betacellulin; (ii) a NEUROD1 inducer; (iii) a G protein-coupled estrogen receptor 1 (GPER) agonist; (iv) a histone methyltransferase inhibitor; (v) an aurora kinase inhibitor; (vi) a ROCK inhibitor; and (vii) a pan-ErbB inhibitor.

2. The method of claim 1, wherein the growth factor is selected from the group consisting of epidermal growth factor (EGF), hepatocyte growth factor (HGF), keratinocyte growth factor (KGF) and insulin-like growth factor (IGF)

3. The method of claim 1 or, wherein the Wnt signaling activator is selected from the group consisting of CHIR99021. SB216763, TWS119, CHIR98014, Tideglusib, SB415286, LY2090314, CHIR-98014, AZD1080. TDZD-8 and wnt3a.

4. The method of any one of claims 1 3 claim 1, wherein the TGFβ family member is Activin-A or nodal.

5. The method of claim 1, wherein the TGFβ receptor (BMP) inhibitor signaling inhibitor is selected from the group consisting of Dorsomorphin, noggin, LDN193189, K 02288, ML347, DMH1, DMH2, LDN 214117 and LDN 212854.

6. The method of claim 1, wherein the sonic hedgehog signaling inhibitor is selected from the group consisting of SANT1, KAAD-cyclopamine, cyclopamine, GANT61, and BMS-833923.

7. The method of claim 1, wherein the PKC activator is selected from the group consisting of TPB ((2S,5S)-(E,E)-8-(5-(4-(trifluoromethyl) phenyl)-2,4-pentadienoylamino)benzolactam); (2/T, 4/T)-L-[(2L′,5U)-1 ,2,3,4,5.6-Hexahydro-5-(hydroxymethyl)-1-methyl-2-(1-methylethyl)-3-oxo-1.4-benzodiazocin-8-yl]-5-[4-(trifluoromethyl) phenyl]-2,4-pentadienamide (TPPB); 5-Chloro-TV-(6-phenylhexyl)-1-naphthalenesulfonamide (SC-9); PDBu; Ingenol 3-angelate (PEP005), and Bryostatin.

8. The method of claim 1, wherein the retinoic acid activator is selected from the group consisting of retinoic acid, TTNPB (4-[(E)-2-(5,6,7.8-Tetrahydro-5.5.8,8-tetramethyl-2-naphthalenyl)-1-propenyl]benzoic acid), EC23, AM580 (4-[(5,6,7,8-Tetrahydro-5.5,8,8-tetramethyl-2-naphthalenyl)carboxamido]benzoic acid), AM 80 and Ch55 (4-[(1E)-3-[3,5-bis(1,1-Dimethylethyl)phenyl]-3-oxo-1-propenyl]benzoic acid).

9. The method claim 1, wherein the ALK5 inhibitor is selected from the group consisting of RepSox, GW788388, SB431542, and LY2109761.

10. The method claim 1, wherein the RHO/ROCK (Rho-associated, coiled-coil containing protein kinase) inhibitor is selected from the group consisting of Y27632, H1152, Fasudil GSK269962, Blebbistatin, HA1100 and RK11447.

11. The method claim 1, wherein the cyclic AMP activator is selected from the group consisting of forskolin, dbcAMP, 8-Br-CAMP, prostaglandin E2 (PGE2), rolipram, and genistein.

12. The method claim 1, wherein the FGF inhibitor is selected from the group consisting of PD173074, PD 161570, SU 5402, SU 5402, and SU 6668.

13. The method claim 1, wherein the aurora kinase inhibitor is selected from the group consisting of ZM447439, VX 680, PF 03814735, and Hesperadin hydrochloride

14. The method claim 1, wherein the NEUROD1 inducer inhibitor is ISX-9 (N-Cyclopropyl-5-(2-thienyl)-3-isoxazolecarboxamide)

15. The method any one of claims 1 13 claim 1, wherein the GPER agonist is selected from the group consisting of G-1, ICI 182,780, and genistein

16. The method claim 1, wherein the histone methyltransferase inhibitor is selected from the group consisting of Deazaneplanocin-A, Procainamide HCl, GSK503, SGC 0946 and SGI-1027.

17. The method claim 1, wherein the pan-ErbB is selected from the group consisting of Canertinib dihydrochloride, Dacomitinib, AZD8931, EKB-569, Lapatinib, PD 158780 and PKI-166.

18. The method of claim 1. wherein the cell culture medium is further supplemented with one or more compounds selected from the group consisting of vitamin C, ZnSO₄, nicotinamide, thyroid hormone, GABA and betacellullin

19. The method of claim 1, wherein the cell culture medium comprises a compound selected from the group consisting of LDN, T3, RepSox, ZnS04, GSIXX, RA, HGF, IGF1, and PD17307.

20. The method of claim 1, wherein the cell culture medium comprises a compound selected from the group consisting of BTC, ISX-9, G-1, Deza, ZM, H1152 and CI-1033.

21. A cell population comprising cells of pancreatic beta lineage obtained by the method according to any one of claims 1-20.
